(19) United States
(12) Patent Application Publication
(10) Pub. No.: US 2020/0297671 A1
(43) Pub. Date: Sep. 24, 2020

(54) METHODS OF AVOIDING EXCIPIENT-BASED ADVERSE EFFECTS AND OF EXPLOITING BIOLOGICAL PROPERTIES OF GRAS COMPOUNDS

(71) Applicants: MASSACHUSETTS INSTITUTE OF TECHNOLOGY, CAMBRIDGE, MA (US); THE BRIGHAM AND WOMEN'S HOSPITAL, INC., BOSTON, MA (US)

(72) Inventors: ROBERT LANGER, NEWTON, MA (US); CARLO TRAVERSO, NEWTON, MA (US); DANIEL REKER, SOMERVILLE, MA (US); STEVEN BLUM, CAMBRIDGE, MA (US)

(21) Appl. No.: 16/803,577

(22) Filed: Feb. 27, 2020

Related U.S. Application Data
(60) Provisional application No. 62/943,746, filed on Dec. 4, 2019.
(60) Provisional application No. 62/817,518, filed on Mar. 12, 2019.
(60) Provisional application No. 62/811,502, filed on Feb. 27, 2019.

(51) Int. Cl. A61K 31/19; G16H 20/10; G16B 5/00; A61K 31/22; A61K 35/00; A61P 35/00

(52) U.S. Cl. CPC A61K 31/19; G16H 20/10; G16B 5/00; A61K 9/0053; A61K 35/00; A61P 35/00; A61K 31/22

(57) ABSTRACT

This invention relates to methods of selecting or tailoring a therapeutic for an individual subject, reducing the excipient burden in a subject, and identifying adverse reaction-associated inactive ingredients in a subject being administered multiple drugs. The invention also relates to methods of inhibiting UGT2B7 activity or P-gp activity, methods of treating a subject via co-administration of a UGT2B7 inhibitor or a P-gp inhibitor, and pharmaceutical compositions comprising gum rosin, abietic acid, or vitamin A palmate.

### RELATED APPLICATIONS

This application claims the benefit of U.S. Provisional Patent Application No. 62/811,502, filed Feb. 27, 2019; U.S. Provisional Patent Application No. 62/817,518, filed Mar. 12, 2019; and U.S. Provisional Patent Application No. 62/943,746, filed Dec. 4, 2019, the disclosures of which are incorporated by reference herein in their entireties.

### GOVERNMENT LICENSE RIGHTS

this invention was made with government support under Grand No. R37-EB000244 awarded by the National Institutes of Health (NIH). the government has certain rights in the invention.

### BACKGROUND

Oral drug products include both the active pharmaceutical ingredient (API) and a specific mixture of inactive ingredients (excipients). The United States Food and Drug Administration (FDA) defines the API as the compound intended to provide the desired pharmaceutical effect. Conversely, inactive ingredients are broadly defined as “any component of a drug product other than an active ingredient”. These components are not intended or expected to have a direct biological or therapeutic effect but instead are added to alter the physical properties of an oral solid dosage form (tablet or capsule) to facilitate absorption or to improve stability, taste, appearance, or to render the therapeutic tamper-resistant. Together, the API and the inactive ingredients make up a specific pharmaceutical formulation.

Decades of pharmaceutical development have tailored inactive ingredient components to ensure that the desired properties of the formulation are met. Manufacturers will often design formulations by borrowing from thousands of known inactive ingredients because approval of novel excipients can require extensive toxicological profiling. Although established excipients have precedence of showing safety on the population level and can be reviewed to evaluate their toxicities, health effects that are undetectable in current preclinical toxicology screenings could remain obscured. Scattered case reports have brought this to the attention of formulation scientists, clinicians, and legislative agencies, but the magnitude and scope of this challenge is currently unknown. Accordingly, it would be desirable to have an analytical method that would empower clinicians to make conscious selections of formulations focusing on their patients' well-being.

Conversely, many inactive ingredients could have beneficial biological effects that might be currently underappreciated. These could provide prime starting points for drug discovery and as functional foods, given the well-understood safety, metabolism, and pharmacokinetics of such compounds. Furthermore, they might warrant the rational design of functional formulations, which will enable the translation of therapeutics to patients that are currently restricted through limited liberation, absorption, distribution, metabolism, excretion, and toxicity (LADMET) profiles.

### SUMMARY

The disclosure provides a method of selecting a therapeutic for an individual subject, the method comprising the steps of: providing a formulation network that depicts available alternatives of dosage forms and interchangeabilities of functionally equivalent inactive ingredients, selecting a first drug formulation from within the formulation network wherein the first drug formulation comprises an active pharmaceutical ingredient and at least one inactive ingredient, identifying at least one ingredient that is toxic to the individual subject from among the at least one inactive ingredient in the first drug formulation, selecting and administering a second drug formulation from within the formulation network wherein the second drug formulation comprises the active pharmaceutical ingredient and does not comprise the at least one toxic ingredient.

The disclosure also provides a method of treating a subject with a therapeutic comprising an active pharmaceutical ingredient (API) comprising evaluating a first set of excipients provided with an API for toxicity in the subject; replacing the first set of excipients wherein one or more of the excipients is found to be toxic to the subject with a second set of excipients, wherein the toxic excipients in the first set of excipients are replaced with non-toxic excipients that are functionally equivalent to the corresponding toxic excipient; and administering the API with the second set of excipients to the subject.

In one embodiment of the method, the toxicity is an allergy. In another embodiment, the allergy is to gluten or lactose. In another embodiment, the functional equivalence is selected from the group consisting of antiadherence, binding, coating, color, disintegration, flavor, providing glide, lubrication, preservation of the API, prevention of water absorption, sweetening, bulking, vehicles, and bioequivalents. In another embodiment, the one or more excipients found to be toxic to the subject are selected from the group consisting of a food, a polymer, a dye, and a sugar. In another embodiment, the one or more excipients found to be toxic to the subject are selected from the group consisting of lactose, corn starch, PEG, povidone, carboxymethylcellulose, gelatin, Brilliant Blue, Sunset Yellow FCF, Allura Red, propylene glycol, indigo carmine, mannitol, sucrose, sodium benzoate, parabens, aspartame, erythrosine, tartrazine, saccharine, poloxamer, soybean oil, benzyl alcohol, vanilla, castor oil, cetyl alcohol, sulfite, PEG castor oils, peanut oil, benzoic acid, corn syrup, sesame oil, starch wheat, casein, banana essence, milk, glucosamine, new coccine, and stearyl alcohol.

In another embodiment of the method of treating, the toxicity is gastrointestinal distress. In another embodiment, the gastrointestinal distress is caused by a fermentable oligosaccharide, disaccharide, monosaccharide, or polyol (FODMAP).

In another embodiment, the first set of excipients and the second set of excipients have previously been administered to a human.

The disclosure also provides a method of mitigating toxicity in a subject wherein the toxicity is the result of at least one toxic inactive ingredient in a first therapeutic composition, the method comprising providing a formulation network which depicts available alternatives of dosage forms and interchangeabilities of inactive ingredients, identifying the at least one toxic inactive ingredient in the first therapeutic composition, applying the formulation network to identify a second therapeutic composition comprising the same API or a therapeutically-similar API as the first therapeutic composition, wherein the second therapeutic composition comprises at least one inactive ingredient that is functionally equivalent to the at least one toxic inactive ingredient in the first therapeutic composition, and wherein the at least one inactive ingredient of the second therapeutic composition has reduced toxicity in the subject with respect to the at least one toxic ingredient in the first therapeutic composition, and selecting and administering the second therapeutic composition to the subject.

The disclosure also a method of tailoring a therapeutic for an individual subject having an allergy, the method comprising the steps of: providing a formulation network that depicts available alternatives of dosage forms and interchangeabilities of functionally equivalent inactive ingredients, selecting a first drug formulation from within the formulation network wherein the first drug formulation comprises an active pharmaceutical ingredient and at least one inactive ingredient, identifying at least one ingredient that is an allergen to the individual subject from among the at least one inactive ingredient in the first drug formulation, selecting and administering a second drug formulation from within the formulation network wherein the second drug formulation comprises the active pharmaceutical ingredient and does not comprise the at least one allergen.

The disclosure also provides a method of tailoring a pharmacokinetic or metabolic profile of a therapeutic for an individual subject, the method comprising the steps of: providing a formulation network that depicts available alternatives of dosage forms and interchangeabilities of functionally equivalent inactive ingredients, selecting a first drug formulation from within the formulation network wherein the first drug formulation comprises an active pharmaceutical ingredient and at least one inactive ingredient, selecting and administering a second drug formulation from within the formulation network wherein the second drug formulation comprises the active pharmaceutical ingredient and at least one inactive ingredient that contributes to the pharmacokinetic or metabolic profile of the second drug formulation, and wherein the second drug formulation possesses a superior pharmacokinetic or metabolic profile with respect to the first drug formulation.

The disclosure also provides a method of determining the total excipient burden in a subject ingesting multiple drugs, the method comprising: identifying all excipients in the multiple drugs being ingested by the subject; and quantifying the total amount of each excipient being ingested by the subject during a specified timeframe.

The disclosure also provides a method of identifying adverse reaction-associated inactive ingredients (ARAIIs) in a subject ingesting multiple drugs, the method comprising: identifying all excipients in the multiple drugs being ingested by the subject, quantifying the total amount of each excipient being ingested by the subject to determine an excipient burden, identifying one or more symptoms experienced by the subject during administration of the multiple drugs, and correlating the excipient burden to the one or more symptoms to establish a potential causal relationship.

The disclosure also provides a method of selecting a therapeutic for a subject with irritable bowel syndrome, small intestinal bacterial overgrowth, or dyspepsia, the method comprising: identifying a first therapeutic formulation, wherein the first therapeutic formulation comprises an active pharmaceutical ingredient (API) and one or more excipients; identifying an adverse reaction-associated inactive ingredient (ARAII) from among the one or more excipients in the first therapeutic formulation by determining that the ARAII provokes an adverse reaction in the subject's gastrointestinal (GI) tract; and selecting a second therapeutic formulation comprising the API of the first therapeutic formulation and one or more excipients, wherein the one or more excipients of the second therapeutic formulation comprise a reduced amount of the ARAII in the first therapeutic formulation.

In one embodiment of the method of selecting a therapeutic, the method further comprises administering a therapeutically effective amount of the second therapeutic formulation to the subject. In another embodiment, the second therapeutic formulation is administered orally. In another embodiment, the API is selected from the group consisting of a proton pump inhibitor, a histamine 2 blocker, and an irritable bowel syndrome treatment. In another embodiment, the API is selected from the group consisting of omeprazole, lansoprazole, dexlansoprazole, rabeprazole, pantoprazole, esomeprazole, famotidine, cimetidine, nizatidine, ranitidine, hyoscyamine sulfate, dicyclomine, lubiprostone, linaclotide, alosetron, rifaximin, and amitriptyline. In another embodiment, the amount of the ARAII in the second therapeutic formulation is less than 70% of the amount of the ARAII in the first therapeutic formulation. In another embodiment, the ARAII is eliminated from the second therapeutic formulation.

In another embodiment, the ARAII is selected from the group consisting of Allura Red, aspartame, banana, benzoic acid, benzyl alcohol, carboxymethylcellulose calcium, casein, castor oil, cetyl alcohol, starch, corn syrup, Brilliant Blue, indigo carmine, erythrosine, Sunset Yellow FCF, tartrazine, gelatin, glucosamine, lactose, mannitol, milk, new coccine, parabens, peanut oil, PEG castor oils, poloxamer, PEG, povidone, propylene glycol, saccharine, sesame oil, sodium benzoate, soybean oil, starch wheat, stearyl alcohol, sucrose, sodium metabisulfite, and vanilla. In another embodiment, the ARAII is selected from the group consisting of a fermentable oligosaccharide, a disaccharide, a monosaccharide, and a polyol (FODMAP). In anonther embodiment, the FODMAP is selected from the group consisting of lactose, mannitol, and polydextrose.

In another embodiment, the second therapeutic is administered in a therapeutic amount for the treatment of a disorder that is not irritable bowel syndrome, small intestinal bacterial overgrowth, or dyspepsia.

The disclosure also provides a method of reducing the total adverse reaction-associated inactive ingredient (ARAII) excipient burden in a subject being administered multiple drugs, the method comprising: identifying a set of therapeutics being administered to the subject; identifying excipients and APIs in the set of therapeutics being administered to the subject; identifying an excipient being administered to the subject as the ARAII in the subject; quantifying the total amount of the ARAII being administered to the subject to determine a first excipient burden; and selecting a new set of therapeutics wherein the new set of therapeutics comprises the APIs of the first set of therapeutics and wherein the new set of therapeutics comprises a second excipient burden that is less than the first excipient burden.

In one embodiment of the method of reducing the total ARAII excipient burden, the method further comprises administering the new set of therapeutics to the subject.

In another embodiment, selection of the new set of therapeutics is performed using a formulation network that depicts available alternatives of dosage forms and interchangeabilities of functionally equivalent excipients, wherein (i) the formulation network comprises at least two nodes; (ii) each node corresponds to a unique therapeutic formulation comprising an API and one or more excipients; and (iii) any two nodes corresponding to two interchangeable therapeutic formulations comprising the same API are connected to each other with an edge.

The disclosure also provides a method of designing a therapeutic formulation, the method comprising: identifying a first therapeutic formulation comprising a first API and one or more excipients, wherein the first therapeutic formulation has previously been administered to a human; identifying a second API that is structurally similar to the first API; and combining the one or more excipients of the first therapeutic formulation with the second API to arrive at a second therapeutic formulation.

In one embodiment of the method of designing a therapeutic formulation, the one or more excipients do not comprise an ARAII. In another embodiment, the second API has previously been administered to a human in at least one therapeutic formulation comprising an ARAII. In another embodiment, the second therapeutic formulation is not commercially available.

The disclosure also provides a method of inhibiting UGT2B7 activity, comprising contacting a cell having UGT2B7 activity with gum rosin. The disclosure additionally provides a method of inhibiting UGT2B7 activity, comprising contacting a cell having UGT2B7 activity with abietic acid.

The disclosure also provides a method of treating a disease or disorder in a subject in need thereof, comprising co-administering to the subject: (1) an effective amount of an active pharmaceutical ingredient (API), wherein the API undergoes UGT2B7-mediated glucuronidation, and (2) a UGT2B7 inhibitor selected from the group consisting of gum rosin and abietic acid.

In one embodiment of the method, the UGT2B7 inhibitor and the API are co-administered in a formulation wherein the UGT2B7 inhibitor and the API are mixed together. In another embodiment, the UGT2B7 inhibitor is not used as a coating.

In another embodiment of the method, the API is selected from the group consisting of: hydromorphone, losartan, diclofenac, etodolac, flurbiprofen, ibuprofen, naproxen, suprofen, mitiglinide, zaltoprofen, ambrisentan, troglitazone, morphine, indomethacin, mycophenolate mofetil, ezetimibe, mycophenolic acid, vadimezan, epirubicin, tapentadol, pitavastatin, silodosin, zidovudine, lovastatin, simvastatin, oxazepam, carbamazepine, codeine, fluvastatin, valproic acid, dapagliflozin, enasidenib, nalmefene, acemetacin, ertugliflozin, artenimol, labetalol, tamoxifen, carvedilol, ketorolac, dabigatran etexilate, dexibuprofen, gemfibrozil, anastrozole, and loxoprofen.

The disclosure also provides a method of reducing the dose of a UGB2B7-sensitive API in a patient population being treated with the API comprising co-administering to the patient population: (1) a UGT2B7 inhibitor selected from the group consisting of gum rosin and abietic acid, and (2) an effective amount of the API, wherein the effective amount of the API being co-administered is lower than the effective amount required to induce the same therapeutic effect in the absence of the UGT2B7 inhibitor.

The disclosure also provides a method of inhibiting P-glycoprotein activity, comprising contacting a cell having P-glycoprotein activity with vitamin A palmitate.

In one embodiment of the method, the cell overexpresses P-glycoprotein.

The disclosure also provides a method of treating cancer in a subject in need thereof comprising co-administering to the subject: (1) an effective amount of one or more chemotherapeutic agents, and (2) vitamin A palmitate.

In one embodiment of the method, the cancer is characterized by P-glycoprotein overexpression. In another embodiment, the cancer is multidrug-resistant cancer.

In another embodiment of the method, the chemotherapeutic is selected from the group consisting of alkylating agents, tumor necrosis factors, intercalators, microtubulin inhibitors, topisomerase inhibitors, and tyrosine kinase inhibitors. In yet another embodiment, the one or more chemotherapeutic agents have increased cell permeability when co-administered with vitamin A palmitate compared to administration of the one or more chemotherapeutic agents without vitamin A palmitate.

The disclosure also provides a pharmaceutical composition comprising: (1) an active pharmaceutical ingredient (API), wherein the API undergoes UGT2B7-mediated glucuronidation, and (2) a UGT2B7 inhibitor selected from the group consisting of gum rosin and abietic acid.

In one embodiment of the pharmaceutical composition, the API and the UGT2B7 inhibitor are co-formulated as a mixture. In another embodiment, the UGT2B7 inhibitor is not used as a coating.

The disclosure additionally provides a pharmaceutical composition comprising a chemotherapeutic agent and vitamin A palmitate.

In one embodiment of the pharmaceutical composition, the chemotherapeutic agent is a P-gp substrate.

### BRIEF DESCRIPTION OF THE FIGURES

FIG. 1 is a graph presenting the number of publications in PubMed containing the search terms “excipient allergy” (circles) or “excipient irritation” (triangles) per year.

FIG. 2 is a pie chart presenting the percentage of the mass of a medication corresponding to inactive versus active ingredients.

FIG. 3 is a graph presenting a correlation analysis between the mass and percentage of inactive ingredients in a given medication. Shading inside circles denotes dose. Different formulations for the same API and dose are grouped together (valsartan 40 mg (I), cyclosporine 100 mg (II), and amoxicillin 1 g (III)).

FIG. 4 is a graph presenting the distribution of inactive ingredients in oral solid dosage forms. The median (eight) is highlighted. Insert shows the distribution of 596 formulations with 20 inactive ingredients or more

FIG. 5 is a graph presenting the frequency of specific inactive ingredients. Gini coefficient=0.95.

FIG. 6 is a graph presenting the formulation heterogeneity for the 18 most-prescribed single-agent oral medications during 2016. Triangles denote the number of different available formulations; the mean and standard-deviation of the distribution of the number of inactive ingredients contained in these formulations are depicted by circles and lines, respectively.

FIG. 7A is a formulation network highlighting complexity of formulation space. Each node corresponds to a specific combination of inactive ingredients; two nodes are connected when at least one API has been commercially formulated with each of these separate combinations of inactive ingredients. Node color corresponds to frequency of formulation usage, edge thickness corresponds to number of APIs that have been formulated with either of the two inactive ingredient combinations. Few clusters of inactive ingredients are exclusively applied to certain drugs (periphery), whereas other formulations are heavily applied to many different APIs and form a complex relationship (central region). FIG. 7B is an enlarged valproic acid region from FIG. 7A showing a network for three different combinations of inactive ingredients currently used to formulate valproic acid. Darker shading indicates more frequent use.

FIG. 8 is a pie chart depicting percentage of medications containing potential allergen classes. From the innermost ring going outwards, the allergen classes are foods, polymers, dyes, sugars, others, and no allergens.

FIG. 9A is a series of pie charts showcasing the percentage of drugs where all formulations contain at least one allergen from the allergen ingredient classes (black), drugs where all available medications are free of such potentially allergy-inducing inactive ingredients belonging to those classes (dark gray), and drugs where some but not all formulations contain at least one ingredient from these classes (light gray). FIG. 9B is a graph showing overall potential allergen content in different formulations of active ingredients. A total of 72% of APIs have all their medications contain at least one of these allergy-associated inactive ingredients (black bar). Medications for 12% of APIs are completely free of concerning inactive ingredients (dark grey). Medications for 16% of APIs have at least one allergen-free formulation (light grey).

FIG. 10 is a graph presenting the percentage of APIs with potential allergens. Black bar: all formulations of the API contain a specific allergy-associated inactive ingredient; dark gray: all formulations of the API are devoid of the allergen inactive ingredient; light gray: some formulations of the API contain the potential allergen.

FIG. 11 is a heatmap showing the ARAII content of different GI therapeutics, grouped by medication class. Numbers in parentheses indicate number of available formulations; PPI: proton pump inhibitor, H2B: Histamine 2 blockers, IBS: irritable bowel syndrome treatments.

FIG. 12 is a graph presenting an analysis of FODMAP content in selected gastrointestinal therapeutics.

FIG. 13 is a graph representing analysis of lactose content in selected gastrointestinal therapeutics.

FIG. 14 is a set of graphs showing the distribution of molecular weight, calculated logP and the fraction of rotational bonds among GRAS compounds and inactive ingredients compared to FDA-approved drugs in the Drugbank database.

FIG. 15 is a visualization of chemical space spanned by GRAS compounds and inactive ingredients (light circles) compared to FDA-approved drugs in the Drugbank database (dark circles).

FIG. 16 is a pharmacology network of GRAS compounds and inactive ingredients. Compounds are shown as light circles and protein targets are shown as dark circles. A compound and a target are connected either when the compound has been previously measured to interact with the protein (black edge) or when machine learning models predict that the compound is likely to interact with the protein (gray edge).

FIG. 17 is a set of pie charts showing the distribution of a number of previously reported (top) and computationally predicted (bottom) activities on the level of different protein families (inner pie chart). The outer pie chart visualizes the number of reported or predicted activities per protein.

FIG. 18 is a graph showing that gum rosin (circles) and abietic acid (squares) can inhibit UGT2B7 activity in microsomes.

FIG. 19 is a graph showing the effect of abietic acid on UGT activity in complex tissue liver lysates.

FIG. 20 is a docking pose indicating that abietic acid has the potential to interact with UGT2B7 at the interface of the substrate- and co-factor-binding domains.

FIG. 21 is a graph showing that vitamin A palmitate inhibits P-gp activity in HepG cells with an IC₅₀ of 3.8 μM.

FIG. 22 is a graph showing that vitamin A palmitate increases the permeability of the P-gp substrates irinotecan, ranitidine, colchicine, and loperamide across porcine intestinal tissue.

FIG. 23 is a graph indicating that vitamin A palmitate induces an increase of systemic warfarin, a known P-gp substrate, after oral delivery in mice.

FIG. 24 is a docking pose indicating that vitamin A palmitate can bind the ATPase site of P-gp with a stabilizing hydrogen bond formed with Arg-1047.

FIG. 25 is a graph showing the broad range of drugs P-gp is capable of transporting (DrugBank 5.0).

### DETAILED DESCRIPTION

### Definitions

It is to be understood that the terminology used herein is for the purpose of describing particular embodiments only, and is not intended to be limiting. As used in this specification and the appended claims, the singular forms “a,” “an,” and “the” include plural referents unless the context clearly dictates otherwise. Thus, for example, reference to “an excipient” includes a combination of two or more such excipients, reference to “an active pharmaceutical ingredient” includes one or more active pharmaceutical ingredients, and the like. Unless specifically stated or obvious from context, as used herein, the term “or” is understood to be inclusive and covers both “or” and “and.”

Unless defined otherwise, all technical and scientific terms used herein have the same meaning as commonly understood by one of ordinary skill in the art to which the invention pertains. Although other methods, systems, and networks similar, or equivalent, to those described herein can be used in the practice of the present invention, the preferred materials and methods are described herein.

In describing and claiming the present invention, the following terminology will be used in accordance with the definitions set out below.

The terms “active pharmaceutical ingredient” and “API” as used herein refer to any substance or mixture of substances intended to be used in the manufacture of a therapeutic product and that, when used in the production of a therapeutic, becomes an active ingredient of the therapeutic product. Such substances are intended to furnish pharmacological activity or other direct effects in the diagnosis, cure, mitigation, treatment, or prevention of disease or to affect the structure and function of the body. Active pharmaceutical ingredients include, but are not limited to, small molecules, peptides, proteins, antibodies, and combinations thereof.

The terms “administer” and “administering” as used herein refer to the providing a therapeutic to a subject. Multiple techniques of administering a therapeutic exist in the art including, but not limited to, intravenous, oral, aerosol, parenteral, ophthalmic, pulmonary, and topical administration.

The term “co-administer” or “ co-administration” or the like as used herein are meant to encompass administration of the selected agents to a single subject, and are intended to include treatment regimens in which the agents are not necessarily administered by the same route of administration or at the same time.

The terms “adverse reaction-associated inactive ingredient” and “ARAII” as used herein refer to excipients or inactive ingredients used in a therapeutic formulation that may cause an undesired response in a subject. The undesired response may, by nonlimiting example, be an allergic reaction or gastrointestinal distress.

The term “allergy” as used herein refers to an immunologically mediated response to a substance in a sensitized subject.

The terms “antiadherent” and “antiadherence” as used herein refer to the property of a substance or to a substance that, when added to a therapeutic, reduces the adhesion between the powdered form of the therapeutic and the components of a tablet press (especially the punch faces), thereby preventing sticking to the tablet press. Antiadherents may also protect tablets from sticking to one another.

The terms “bulking” or “bulking agent” as used herein refer to the property of a substance or to a substance that, when added to a therapeutic, increases the bulk or mass of the therapeutic.

The terms “binding” and “binder” as used herein refer to the property of a substance or to a substance that, when added to a therapeutic, facilitates cohesion of the components therein. Binders may be solution binders (i.e., dissolved in a solvent) or dry binders (i.e., added to a powder blend).

The term “coating” as used herein refers to a substance which acts as a barrier on the surface of a tablet, capsule, or the like to protect the ingredients therein from atmospheric moisture; to make an unpleasant-tasting tablet, capsule, or the like easier to swallow; or to protect the ingredients in the tablet, capsule, or the like from the acidic conditions of the gastrointestinal tract. Non-limiting examples of coatings include stearic acid, beeswax, carnauba wax, shellac, crystalline wax, lanolin, paraffin, gum arabic, guar gum, gum rosin, and abietic acid.

The terms “color” or “dye” as used herein refer to the property of a substance or to a substance that, when added to a therapeutic, alters the visual quality of the therapeutic with respect to hue, saturation, and brightness of reflected light. A dye may be used to affect the aesthetic look of a therapeutic.

The terms “disintegration” or “disintegrant” as used herein refer to the property of a substance or to a substance that, when added to a therapeutic (especially a tablet), causes the therapeutic to break apart when exposed to an aqueous environment by expanding or dissolving when wet, thereby facilitating the release of the active pharmaceutical ingredients for absorption.

The terms “drug,” “drug formulation,” “formulation,” “therapeutic,” “therapeutic formulation,” “therapeutic product,” and the like are used interchangeably herein to refer to any composition which is suitable for administration to a subject and which comprises at least one active pharmaceutical ingredient and at least one excipient. Such substances are biologically, physiologically, or pharmacologically active in a subject, locally and/or systemically.

The terms “excipient” and “inactive ingredient” are used interchangeably herein refer to any component of a drug product other than the active pharmaceutical ingredient that may be used to modify or improve the drug release, improve its physical and/or chemical stability, dosage form performance, processing, manufacturing, etc. Excipients include, but are not limited to, fillers, solvents, dispersion media, diluents, coatings, antibacterial and antifungal agents, isotonic and absorption-delaying agents, etc.

The term “excipient burden” as used herein refers to the total amount of an excipient or inactive ingredient being administered to a subject from all drugs being administered to the same subject over a specific period of time. In certain embodiments, the excipient burden is the ARAII excipient burden. As used herein, this term refers to the total amount of ARAII excipient inactive ingredient being administered to a subject from all drugs being administered to the same subject over a specific period of time.

The terms “flavor” or “flavoring” as used herein refer to the property of a substance or to a substance that, when added to a therapeutic alters the distinctive taste of the therapeutic. Flavors may be added to a therapeutic in order to mask unpleasant-tasting ingredients used therein. Flavors may be natural or artificial.

The terms “functionally equivalent” and “functional equivalent” as used herein refer to the quality of two or more substances to perform the same function.

The terms “glide” and “glidant” as used herein refer to the property of a substance or to a substance that, when added to a therapeutic, improves the flowability of the powder form of the therapeutic. A glidant may function by, for example, reducing interparticle friction or decreasing surface charge.

The terms “lubrication” and “lubricant” as used herein refer to the property of a substance or to a substance that, when added to a therapeutic, reduces friction at the interface between a tablet's surface and the die wall of a tablet press during ejection, thereby reducing wear on the components of the tablet press.

The terms “preservation of the API” and “preservative” as used herein refer to the property of a substance or to a substance that, when added to a therapeutic, prevents physical or chemical decomposition of said therapeutic. Preservatives may, by nonlimiting example, be antimicrobial agents or antioxidants.

The term “prevention of water absorption” as used herein refers to the property of an agent to impede or avert the incorporation of water (e.g., in a therapeutic product).

The term “previously administered to a human” and iterations thereof as used herein refers to substances, drugs, formulations, and compositions which have been administered to at least one human subject. The substances, drugs, formulations, and compositions previously administered to a human may or may not be commercially available.

The term “subject” as used herein refers to any member of the subphylum Chordata, including, without limitation, humans and other primates, including non-human primates such as rhesus macaques and other monkey species and chimpanzees and other ape species; farm animals such as cattle, sheep, pigs, goats, and horses; domestic mammals such as dogs and cats; laboratory animals including rodents such as mice, rats, and guinea pigs; birds, including domestic, wild, and game birds such as chickens, turkeys, and other gallinaceous birds, ducks, geese, and the like. The term does not denote a particular age or gender. Thus, both adult and newborn individuals are intended to be covered.

The terms “sweetening” or “sweetener” as used herein refer to the property of a substance or to a substance that, when added to a therapeutic, increases the sweetness of the formulation.

The term “structurally similar” as used herein refers to the structural similarity of a first substance with a first chemical structure to a second substance with a second chemical structure. In certain embodiments, when two chemical structures are chemically similar they score greater than 70, 75, 80, 85, 90 or 95% using the Tanimoto algorithm.

The term “therapeutically effective amount” as used herein refers to an amount of a drug, formulation, or composition to achieve a particular biological result. In certain embodiments, the result is the improvement of at least one symptom of a pathology in a subject administered the drug, formulation or composition.

The term “toxic” as used herein refers to the property of a substance to incur adverse effects in the body of a subject wherein the adverse effect is the result of accumulation of said substance in the body.

The term “toxicity” as used herein refers to the quality of being toxic.

The term “vehicle” as used herein refers to the bulk excipient used as a medium for conveying the active pharmaceutical ingredient.

### Methods of Reducing ARAIIs in Therapeutic Formulations

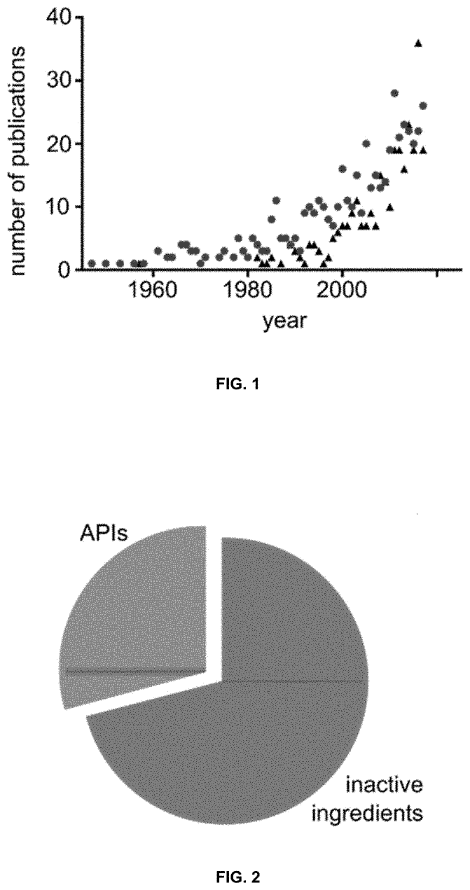

Increasing numbers of clinical reports have documented adverse reactions triggered by an inactive ingredient in a medication (FIG. 1). These adverse reaction-associated inactive ingredients (ARAIIs) can commonly cause symptoms in the form of an allergy or an intolerance. Many allergic reactions to inactive ingredients are Type I hypersensitivity reactions, mediated by Immunoglobulin E recognition of an antigen and characterized by symptoms associated with histamine release such as urticaria, angioedema, bronchospasm, and anaphylaxis. Such rare effects can lead to drastic adverse events in small patient subpopulations. Conversely, intolerances to an inactive ingredient can cause symptoms through mechanisms such as malabsorption, which causes gastrointestinal symptoms via direct osmotic effects or as a result of their fermentation in the digestive system. These potentially affect a much larger population with more benign symptoms compared to allergic reactions. These pathways might lead to adverse drug effects that affect patients' well-being and adherence to drug regimens if the inactive ingredients are present in sufficient quantities to trigger a reaction.

More than 1000 inactive ingredients, or excipients, can be added to pills and capsules to improve their physical properties. The mass content of individual inactive ingredients in pills or capsules is largely not reported by manufacturers and therefore is not easily accessible to patients and health care providers. For many of the reported allergens and irritants, the distribution of sensitivities among relevant patient populations is sparsely understood. However, for almost every drug and every drug class, alternatives exist that avoid certain inactive ingredients. Appropriate selection for every patient will enable maximization of safety and comfort for patients. Accordingly, the methods described below may serve to assist physicians, patients, and pharmacists in the selection of appropriate therapeutics on an individual-by-individual basis.

Methods of treating a subject with a therapeutic are described in detail in this section. Further, methods of preventing or mitigating adverse effects in a subject being treated with a therapeutic wherein the adverse effects arise from the toxicity of inactive ingredients in the therapeutic composition are described in this section. The various embodiments described herein allow a user to tailor or personalize a therapeutic for an individual subject based on a data network that allows for facile selection and replacement of inactive ingredients in the therapeutic formulation.

In a first aspect, the invention relates to a method of tailoring a therapeutic for an individual subject. In some embodiments, the method entails providing the user with a data network that depicts the distinct relationships between formulations for a given API and highlights the available alternatives to a given formulation.

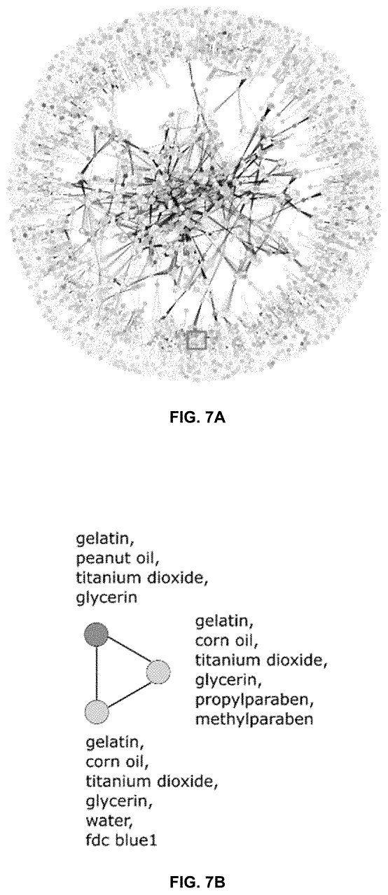

The data network of the invention may be constructed from a database of drug information, for example, from the Pillbox database. In some embodiments, the data network will cluster formulations for a given API together and allow a user to visualize the relationship between different formulations for that API. In some embodiments, the data network will depict available alternatives of all dosage forms for a given API. In some embodiments, the data network will depict interchangeabilities of functionally equivalent inactive ingredients for a given API. In a preferred embodiment, the data network is a formulation network as exemplified in FIG. 7A. In some embodiments, the formulation network comprises a number of nodes corresponding to the number of unique combinations of inactive ingredients for any given API in the database. The formulation network may further comprise edges that connect the nodes to highlight the interchangeability of formulations.

In some embodiments, the method further entails selecting a first drug formulation from within the data network wherein the first drug formulation comprises an API and at least one inactive ingredient.

In some embodiments, the method further entails identifying at least one ingredient that is toxic to the individual subject from among the at least one inactive ingredient in the first drug formulation.

Toxic ingredients may, by nonlimiting example, be foods, polymers, dyes, sugars, or other substances that incur an adverse reaction in an individual subject. In some embodiments, the toxic ingredient identified may be one of lactose, corn starch, PEG, povidone, carboxymethylcellulose, gelatin, Brilliant Blue, Sunset Yellow FCF, Allura Red, propylene glycol, indigo carmine, mannitol, sucrose, sodium benzoate, parabens, aspartame, erythrosine, tartrazine, saccharine, poloxamer, soybean oil, benzyl alcohol, vanilla, castor oil, cetyl alcohol, sulfite, PEG castor oils, peanut oil, benzoic acid, corn syrup, sesame oil, starch wheat, casein, banana essence, milk, glucosamine, new coccine, or stearyl alcohol.

Toxic ingredients may incur an allergic reaction in the subject. The allergic reaction may be a Type I hypersensitivity reaction, mediated by Immunoglobulin E recognition of an antigen and characterized by symptoms associated with histamine release such as urticaria, angioedema, bronchospasm, and anaphylaxis. In some embodiments, the allergic reaction is one to gluten or lactose. In some embodiments, the allergic reaction is a severe allergic reaction. In other embodiments, the toxic ingredient may incur an adverse reaction that is not an allergic reaction. In some embodiments, the toxic ingredient may incur gastrointestinal distress in the subject. In some embodiments, the gastrointestinal distress may be caused by a fermentable oligosaccharide, disaccharide, monosaccharide, or polyol (FODMAP).

In some embodiments, the method further entails selecting and administering a second drug formulation from within the data network wherein the second drug formulation comprises the API and does not comprise the identified toxic ingredient.

The second drug formulation may comprise the API in the same dosage as the first drug formulation. The second drug formulation may comprise the same number of inactive ingredients, fewer inactive ingredients, or more inactive ingredients than the first drug formulation. In preferred embodiments, wherein the data network is a formulation network as exemplified in FIG. 7A, the node corresponding to the second drug formulation may be connected to the node corresponding to the first drug formulation by no more than 5 edges, by no more than 4 edges, by no more than 3 edges, by no more than 2 edges, or by no more than 1 edge.

In a preferred embodiment of the first aspect of the invention, the method of tailoring a therapeutic for an individual subject entails (a) providing a formulation network that depicts available alternatives of dosage forms and interchangeabilities of functionally equivalent inactive ingredients; (b) selecting a first drug formulation from within the formulation network wherein the first drug formulation comprises an active pharmaceutical ingredient and at least one inactive ingredient; (c) identifying at least one ingredient that is toxic to the subject from among the at least one inactive ingredient in the first drug formulation; and (d) selecting and administering a second drug formulation from within the formulation network wherein the second drug formulation comprises the active pharmaceutical ingredient and does not comprise the at least one toxic ingredient.

In a second aspect, the invention relates to a method of treating a subject with a therapeutic comprising an API. In some embodiments, the method entails evaluating a first set of excipients provided with an API for toxicity in the subject.

The toxicity may be an allergy. The allergy may entail a Type I hypersensitivity reaction, mediated by Immunoglobulin E recognition of an antigen and be characterized by symptoms associated with histamine release such as urticaria, angioedema, bronchospasm, and anaphylaxis. In some embodiments, the allergy is one to gluten or lactose. In some embodiments, the allergy is a severe allergy. In other embodiments, the toxicity may involve an adverse reaction that is not an allergy. In some embodiments, the toxicity may be gastrointestinal distress in the subject. In some embodiments, the gastrointestinal distress may be caused by a fermentable oligosaccharide, disaccharide, monosaccharide, or polyol (FODMAP).

In some embodiments, the method further entails replacing the first set of excipients, wherein one or more of the excipients is found to be toxic to the subject, with a second set of excipients, wherein the toxic excipient(s) of the first set of excipients are replaced with non-toxic excipients. In some embodiments, the toxic excipient(s) in the first set of excipients are replaced with non-toxic excipient(s) that are functionally equivalent to the corresponding toxic excipient(s).

In some embodiments, the first set of excipients and the second set of excipients have previously been administered to a human. In some embodiments, a therapeutic formulation comprising the API and the first set of excipients is commercially available. In some embodiments, a therapeutic formulation comprising the API and the second set of excipients is commercially available.

Toxic ingredients of the second aspect of the invention may, by nonlimiting example, be foods, polymers, dyes, sugars, or other substances that incur an adverse reaction in an individual subject. In some embodiments, the toxic ingredient identified may be one of lactose, corn starch, PEG, povidone, carboxymethylcellulose, gelatin, Brilliant Blue, Sunset Yellow FCF, Allura Red, propylene glycol, indigo carmine, mannitol, sucrose, sodium benzoate, parabens, aspartame, erythrosine, tartrazine, saccharine, poloxamer, soybean oil, benzyl alcohol, vanilla, castor oil, cetyl alcohol, sulfite, PEG castor oils, peanut oil, benzoic acid, corn syrup, sesame oil, starch wheat, casein, banana essence, milk, glucosamine, new coccine, or stearyl alcohol.

Functional equivalence categories that excipients may fall under include, but are not limited to, antiadherence, binding, coating, color, disintegration, flavor, glide, lubrication, preservation of the API, prevention of water absorption, sweetening, bulking, vehicles, and bioequivalents. Replacement of one excipient with a functionally equivalent excipient may, for example, entail substituting one coloring agent or dye (i.e., tartrazine) with a different coloring agent (i.e., beta-Carotene or curcumin); substituting one binder (i.e., lactose) with a different binder (i.e., cellulose); substituting one vehicle (i.e., peanut oil) with a different vehicle (i.e., corn oil).

In some embodiments, the method further entails administering the API with the second set of excipients to the subject.

In a preferred embodiment of the second aspect of the invention, the method of treating a subject with a therapeutic comprising an API entails (a) evaluating a first set of excipients provided with an API for toxicity in the subject; (b) replacing the first set of excipients wherein one or more of the excipients is found to be toxic to the subject with a second set of excipients, wherein the toxic excipients in the first set of excipients are replaced with non-toxic excipients that are functionally equivalent to the corresponding toxic excipient; and (c) administering the API with the second set of excipients to the subject.

In a third aspect, the invention relates to a method of mitigating toxicity in a subject wherein the toxicity is the result of at least one toxic inactive ingredient in a therapeutic composition. In some embodiments, the method entails providing a data network that depicts the distinct relationships between formulations for a given API and highlights the available alternatives to a given formulation.

The data network of the invention may be constructed from a database of drug information, for example, from the Pillbox database. In some embodiments, the data network will cluster formulations for a given API together and allow a user to visualize the relationship between different formulations for that API. In some embodiments, the data network will depict available alternatives of all dosage forms for a given API. In some embodiments, the data network will depict interchangeabilities of functionally equivalent inactive ingredients for a given API. In a preferred embodiment, the data network is a formulation network as exemplified in FIG. 7A. In some embodiments, the formulation network comprises a number of nodes corresponding to the number of unique combinations of inactive ingredients for any given API in the database. The formulation network may further comprise edges that connect the nodes to highlight the interchangeability of formulations.

In some embodiments, the method further entails identifying the toxic ingredient in the therapeutic composition.

A toxic ingredient of the third aspect of the invention may, by nonlimiting example, be a food, polymer, dye, sugar, or other substance that incurs an adverse reaction in an individual subject. In some embodiments, the toxic ingredient identified may be one of lactose, corn starch, PEG, povidone, carboxymethylcellulose, gelatin, Brilliant Blue, Sunset Yellow FCF, Allura Red, propylene glycol, indigo carmine, mannitol, sucrose, sodium benzoate, parabens, aspartame, erythrosine, tartrazine, saccharine, poloxamer, soybean oil, benzyl alcohol, vanilla, castor oil, cetyl alcohol, sulfite, PEG castor oils, peanut oil, benzoic acid, corn syrup, sesame oil, starch wheat, casein, banana essence, milk, glucosamine, new coccine, or stearyl alcohol.

In some embodiments, the method further entails applying the formulation network to identify a second therapeutic composition. In some embodiments, the second therapeutic composition is related to the first therapeutic composition in that it comprises the same API or and API that elicits the same or similar therapeutic effect. In some embodiments, the second therapeutic composition comprises at least one inactive ingredient that has reduced toxicity in the subject with respect to a functionally equivalent inactive ingredient in the first therapeutic composition.

The second drug formulation may comprise the API in the same dosage as the first drug formulation. The second drug formulation may comprise the same number of inactive ingredients, fewer inactive ingredients, or more inactive ingredients than the first drug formulation. In preferred embodiments, wherein the data network is a formulation network as exemplified in FIG. 7A, the node corresponding to the second drug formulation may be connected to the node corresponding to the first drug formulation by no more than 5 edges, by no more than 4 edges, by no more than 3 edges, by no more than 2 edges, or by no more than 1 edge.

Functional equivalence categories that excipients may fall under include, but are not limited to, antiadherence, binding, coating, color, disintegration, flavor, glide, lubrication, preservation of the API, prevention of water absorption, sweetening, bulking, vehicles, and bioequivalents.

In some embodiments, the method further entails administering the second therapeutic composition with reduced toxicity to the subject.

In a preferred embodiment of the third aspect of the invention, the method of mitigating toxicity in a subject wherein the toxicity is the result of at least one toxic inactive ingredient in a first therapeutic composition entails (a) providing a formulation network which depicts available alternatives of dosage forms and interchangeabilities of inactive ingredients; (b) identifying the at least one toxic inactive ingredient in the first therapeutic composition; (c) applying the formulation network to identify a second therapeutic composition comprising the same API or a therapeutically-similar API as the first therapeutic composition, wherein the second therapeutic composition comprises at least one inactive ingredient that is functionally equivalent to the at least one toxic inactive ingredient in the first therapeutic composition, and wherein the at least one inactive ingredient of the second therapeutic composition has reduced toxicity in the subject with respect to the at least one toxic ingredient in the first therapeutic composition; and (d) administering the second therapeutic composition to the subject.

In a fourth aspect, the invention relates to a method of tailoring a therapeutic for an individual subject having an allergy. In some embodiments, the method entails providing the user with a data network that depicts the distinct relationships between formulations for a given API and highlights the available alternatives to a given formulation.

The data network of the invention may be constructed from a database of drug information, for example, from the Pillbox database. In some embodiments, the data network will cluster formulations for a given API together and allow a user to visualize the relationship between different formulations for that API. In some embodiments, the data network will depict available alternatives of all dosage forms for a given API. In some embodiments, the data network will depict interchangeabilities of functionally equivalent inactive ingredients for a given API. In a preferred embodiment, the data network is a formulation network as exemplified in FIG. 7A. In some embodiments, the formulation network comprises a number of nodes corresponding to the number of unique combinations of inactive ingredients for any given API in the database. The formulation network may further comprise edges that connect the nodes to highlight the interchangeability of formulations.

In some embodiments, the method further entails selecting a first drug formulation from within the data network wherein the first drug formulation comprises an API and at least one inactive ingredient.

In some embodiments, the method further entails identifying at least one ingredient that is an allergen to the individual subject from among the at least one inactive ingredient in the first drug formulation.

Allergens may, by nonlimiting example, be foods, polymers, dyes, sugars, or other substances that incur an allergic reaction in an individual subject. The allergen may cause a Type I hypersensitivity reaction, mediated by Immunoglobulin E recognition of the allergen and characterized by symptoms associated with histamine release such as urticaria, angioedema, bronchospasm, and anaphylaxis. In some embodiments, the allergen is one of gluten or lactose. In some embodiments, the allergen may cause a severe allergic reaction in the subject.

In some embodiments, the method further entails selecting and administering a second drug formulation from within the data network wherein the second drug formulation comprises the API and does not comprise the identified allergen.

The second drug formulation may comprise the API in the same dosage as the first drug formulation. The second drug formulation may comprise the same number of inactive ingredients, fewer inactive ingredients, or more inactive ingredients than the first drug formulation. In preferred embodiments, wherein the data network is a formulation network as exemplified in FIG. 7A, the node corresponding to the second drug formulation may be connected to the node corresponding to the first drug formulation by no more than 5 edges, by no more than 4 edges, by no more than 3 edges, by no more than 2 edges, or by no more than 1 edge.

In a preferred embodiment of the fourth aspect of the invention, the method of tailoring a therapeutic for an individual subject having an allergy entails (a) providing a formulation network that depicts available alternatives of dosage forms and interchangeabilities of functionally equivalent inactive ingredients; (b) selecting a first drug formulation from within the formulation network wherein the first drug formulation comprises an active pharmaceutical ingredient and at least one inactive ingredient; (c) identifying at least one ingredient that is an allergen to the subject from among the at least one inactive ingredient in the first drug formulation; and (d) selecting and administering a second drug formulation from within the formulation network wherein the second drug formulation comprises the active pharmaceutical ingredient and does not comprise the at least one allergen.

In a fifth aspect, the invention relates to a method of tailoring the pharmacokinetic or metabolic profiles of a therapeutic for an individual subject. It is known that a few select excipients have the potential to alter the pharmacokinetic properties of an API, for example, via physicochemical interactions or by modulating metabolic and transport enzymes. Appropriate tailoring of a specific formulation for a specific patient could thereby allow for fine-tuned pharmacokinetic and metabolic profiles.

In some embodiments, the method entails providing the user with a data network that depicts the distinct relationships between formulations for a given API and highlights the available alternatives to a given formulation.

The data network of the invention may be constructed from a database of drug information, for example, from the Pillbox database. In some embodiments, the data network will cluster formulations for a given API together and allow a user to visualize the relationship between different formulations for that API. In some embodiments, the data network will depict available alternatives of all dosage forms for a given API. In some embodiments, the data network will depict interchangeabilities of functionally equivalent inactive ingredients for a given API. In a preferred embodiment, the data network is a formulation network as exemplified in FIG. 7A. In some embodiments, the formulation network comprises a number of nodes corresponding to the number of unique combinations of inactive ingredients for any given API in the database. The formulation network may further comprise edges that connect the nodes to highlight the interchangeability of formulations.

In some embodiments, the method further entails selecting a first drug formulation from within the data network wherein the first drug formulation comprises an API and at least one inactive ingredient.

In some embodiments, the method further entails selecting and administering a second drug formulation from within the data network. In some embodiments, the second drug formulation comprises the API and at least one inactive ingredient that contributes to the pharmacokinetic or metabolic profile of the second drug formulation. In some embodiments, the second drug formulation possesses a superior pharmacokinetic or metabolic profile with respect to the first drug formulation.

The second drug formulation may comprise the API in the same dosage as the first drug formulation. The second drug formulation may comprise the same number of inactive ingredients, fewer inactive ingredients, or more inactive ingredients than the first drug formulation. In preferred embodiments, wherein the data network is a formulation network as exemplified in FIG. 7A, the node corresponding to the second drug formulation may be connected to the node corresponding to the first drug formulation by no more than 5 edges, by no more than 4 edges, by no more than 3 edges, by no more than 2 edges, or by no more than 1 edge.

In a preferred embodiment of the fifth aspect of the invention, the method of tailoring the pharmacokinetic or metabolic profiles of a therapeutic for an individual subject entails (a) providing a formulation network that depicts available alternatives of dosage forms and interchangeabilities of functionally equivalent inactive ingredients; (b) selecting a first drug formulation from within the formulation network wherein the first drug formulation comprises an active pharmaceutical ingredient; and (c) selecting and administering a second drug formulation from within the formulation network wherein the second drug formulation comprises the active pharmaceutical ingredient and at least one inactive ingredient that contributes to the pharmacokinetic or metabolic profile of the second drug formulation, and wherein the second drug formulation possesses a superior pharmacokinetic or metabolic profile with respect to the first drug formulation.

In a sixth aspect, the invention relates to a method of selecting a therapeutic to administer to a subject from a formulation network wherein the formulation network depicts the distinct relationships between formulations for a given API and identifies the available alternatives to a given formulation. In some embodiments, the method entails identifying allergies or sensitivities in the subject. In some embodiments, the method further entails selecting a formulation for the API wherein the formulation does not comprise inactive ingredients that may cause an allergy or adverse reaction in the subject. In some embodiments, the formulation network may be incorporated into a user-friendly interface such as a mobile app.

In a seventh aspect, the invention relates to a method of determining the total excipient burden in a subject ingesting multiple drugs. In some embodiments, the method may entail identifying all the excipients present in the multiple drugs being ingested by the subject. In some embodiments, the method may further entail quantifying the total amount of each excipient being ingested by the subject during a specified timeframe.

In an eight aspect, the invention relates to a method of identifying adverse reaction-associated inactive ingredients (ARAIIs) in a subject ingesting multiple drugs. In some embodiments, the method entails identifying all excipients in the multiple drugs being ingested by the subject. In some embodiments, the method further entails quantifying the total amount of each excipient being ingested by the subject to determine an excipient burden. In some embodiments, the method further entails identifying one or more symptoms experienced by the subject during administration of the multiple drugs. In some embodiments, the method further entails correlating the excipient burden to the one or more symptoms to establish a potential causal relationship.

In a ninth aspect, the invention relates to a method of regulating or reducing the excipient burden in subject ingesting multiple drugs. Previously described embodiments of the invention may be used to identify the excipient burden in a subject and enable a user to select alternate drugs to administer to the subject in order to reduce or eliminate the excipient burden while maintaining previously administered doses of active pharmaceutical ingredients. In one aspect, the method may entail correlating the excipient burden to an allergy, the subject's past medical history, or the subject's general medical record in order to determine favorable formulations.

In a tenth aspect, the invention relates to a method of selecting a drug to administer to a subject. In some embodiments, the subject has been administered a drug comprising an API and an ARAII. In some embodiments, a drug is selected for administration that comprises a different API and further does not comprise an ARAII.

In an eleventh aspect, the invention relates to a method of selecting a therapeutic for a subject with irritable bowel syndrome, small intestinal bacterial overgrown, or dyspepsia. In some embodiments, the method entails identifying a first therapeutic formulation, wherein the first therapeutic formulation comprises an active pharmaceutical ingredient (API) and one or more excipients.

In some embodiments, the first therapeutic formulation may have been administered in the past or may currently be administered to the subject. In some embodiments, the first therapeutic formulation may have been administered orally.

In some embodiments, the API is administered to treat a disease or disorder of the gastrointestinal tract. In some embodiments, the API is a proton pump inhibitor. Proton pump inhibitors include, by nonlimiting example, omeprazole, lansoprazole, dexlansoprazole, rabeprazole, pantoprazole, and esomeprazole. In some embodiments, the API is a histamine 2 blocker. Histamine 2 blockers include, by nonlimiting example, famotidine, cimetidine, nizatidine, and ranitidine. In some embodiment, the API treats irritable bowel syndrome (IBS). IBS treatments include, by nonlimiting example, hyoscyamine sulfate, dicyclomine, lubiprostone, linaclotide, alosetron, rifaximin, and amitriptyline.

In some embodiments, the API is administered to treat a disease or disorder that is not a gastrointestinal disease or disorder. In some embodiments, the API is administered to treat a disorder that is not irritable bowel syndrome, small intestinal bacterial overgrowth, or dyspepsia.

In some embodiments, the method further entails identifying an adverse reaction- associated inactive ingredient (ARAII) from among the one or more excipients in the first therapeutic formulation by determining that the ARAII provokes an adverse reaction in the subject's gastrointestinal (GI) tract.

Example ARAIIs include, but are not limited to, Allura Red, aspartame, banana, benzoic acid, benzyl alcohol, carboxymethylcellulose calcium, casein, castor oil, cetyl alcohol, starch, corn syrup, Brilliant Blue, indigo carmine, erythrosine, Sunset Yellow FCF, tartrazine, gelatin, glucosamine, lactose, mannitol, milk, new coccine, parabens, peanut oil, PEG castor oils, poloxamer, PEG, povidone, propylene glycol, saccharine, sesame oil, sodium benzoate, soybean oil, starch wheat, stearyl alcohol, sucrose, sodium metabisulfite, and vanilla. The ARAII may be a fermentable oligosaccharide, disaccharaide, monosaccharide, or polyol (FODMAP). In some embodiments, the ARAII is a FODMAP selected from the group consisting of lactose, mannitol, and polydextrose.

In some embodiments, the method further entails selecting a second therapeutic formulation comprising the API of the first therapeutic formulation and one or more excipients, wherein the one or more excipients of the second therapeutic formulation comprise a reduced amount of the ARAII in the first therapeutic formulation.

In some embodiments, the amount of the ARAII that would be administered to a subject during a specific timeframe from administration of the second therapeutic formulation is less than 90%, less than 80%, less than 70%, less than 60%, less than 50%, less than 40%, less than 30%, less than 20%, or less than 10% the amount that would be administered to the subject during the same timeframe from administration of the first therapeutic formulation. In a preferred embodiment, the amount of the ARAII that would be administered to a subject from administration of the second therapeutic formulation is less than 70% the amount that would be administered to the subject from administration of the first therapeutic formulation. In some embodiments, the ARAII is eliminated from the second therapeutic formulation.

In some embodiments, the method further entails administering a therapeutically effective amount of the second therapeutic formulation to the subject. In some embodiments, the second therapeutic formulation is administered orally.

In a preferred embodiment of the eleventh aspect of the invention, the method of selecting a therapeutic for a subject with irritable bowel syndrome, small intestinal bacterial overgrowth, or dyspepsia, comprises (a) identifying a first therapeutic formulation, wherein the first therapeutic formulation comprises an active pharmaceutical ingredient (API) and one or more excipients; (b) identifying an adverse reaction-associated inactive ingredient (ARAII) from among the one or more excipients in the first therapeutic formulation by determining that the ARAII provokes an adverse reaction in the subject's gastrointestinal (GI) tract; and (c) selecting a second therapeutic formulation comprising the API of the first therapeutic formulation and one or more excipients, wherein the one or more excipients of the second therapeutic formulation comprise a reduced amount of the ARAII in the first therapeutic formulation.

In a twelfth aspect, the invention relates to a method of reducing the total adverse reaction-associated inactive ingredient (ARAII) excipient burden in a subject being administered multiple drugs. In some embodiments, the method entails identifying a set of therapeutics being administered to the subject.

In some embodiments, the subject has an allergy. In some embodiments, the subject has a gastrointestinal disease or disorder. The gastrointestinal disease or disorder may, by nonlimiting example, be irritable bowel syndrome, small intestinal bacterial overgrowth, or dyspepsia. In some embodiments, at least one of the therapeutics is being administered orally.

In some embodiments, the method further entails identifying excipients and APIs in the set of therapeutics being administered to the subject. In some embodiments, the same excipient may be present in more than one drug being administered to the subject. Excipients may, by nonlimiting example, be antiadherents, bulking agents, binders, coatings, dyes, disintegrants, flavorings, glidants, lubricants, sweeteners, or vehicles.

In some embodiments, the method further entails identifying an excipient being administered to the subject as the ARAII in the subject.

In some embodiments, the ARAII may provoke an allergic reaction in the subject. In some embodiments, the ARAII may provoke an adverse reaction in the subject's gastrointestinal (GI) tract. Example ARAIIs include, but are not limited to, Allura Red, aspartame, banana, benzoic acid, benzyl alcohol, carboxymethylcellulose calcium, casein, castor oil, cetyl alcohol, starch, corn syrup, Brilliant Blue, indigo carmine, erythrosine, Sunset Yellow FCF, tartrazine, gelatin, glucosamine, lactose, mannitol, milk, new coccine, parabens, peanut oil, PEG castor oils, poloxamer, PEG, povidone, propylene glycol, saccharine, sesame oil, sodium benzoate, soybean oil, starch wheat, stearyl alcohol, sucrose, sodium metabisulfite, and vanilla. The ARAII may be a fermentable oligosaccharide, disaccharaide, monosaccharide, or polyol (FODMAP). In some embodiments, the ARAII is a FODMAP selected from the group consisting of lactose, mannitol, and polydextrose.

In some embodiments, the method further entails quantifying the total amount of the ARAII being administered to the subject to determine a first excipient burden. In some embodiments, the total amount of the ARAII being administered is determined over the course of a specific time period. The total amount being administered may, for example, be determined over a timeframe of 12 hours, over a timeframe of 24 hours, over a timeframe of 48 hours, or over a timeframe of 72 hours.

In some embodiments, the method further entails selecting a new set of therapeutics wherein the new set of therapeutics comprises the APIs of the first set of therapeutics and wherein the new set of therapeutics comprises a second excipient burden that is less than the first excipient burden.

In some embodiments, selection of the new set of therapeutics is performed using a formulation network that depicts available alternatives of dosage forms and interchangeabilities of functionally equivalent excipients, as exemplified in FIG. 7A. The formulation network may be constructed from a database of drug information, for example, from the Pillbox database. The formulation network may cluster formulations for a given API together and allow a user to visualize the relationship between different formulations for that API and highlight the interchangeability of formulations. In a preferred embodiment, the formulation network comprises at least two nodes wherein each node corresponds to a unique therapeutic formulation comprising an API and one or more excipients and wherein any two nodes corresponding to two interchangeable therapeutic formulations comprising the same API are connected to each other with an edge.

In some embodiments, the amount of the ARAII that would be administered to a subject during a specific timeframe from administration of the new set of therapeutics is less than 90%, less than 80%, less than 70%, less than 60%, less than 50%, less than 40%, less than 30%, less than 20%, or less than 10% the amount that would be administered to the subject during the same timeframe from administration of the original set of therapeutics. In a preferred embodiment, the amount of the ARAII that would be administered to a subject from administration of the new set of therapeutics is less than 70% the amount that would be administered to the subject from administration of the original set of therapeutics. In some embodiments, the ARAII is eliminated from the new set of therapeutics.

In some embodiments, the method further comprises administering the new set of therapeutics to the subject.

In a preferred embodiment of the twelfth aspect of the invention, the method of reducing the total adverse reaction-associated inactive ingredient (ARAII) excipient burden in a subject being administered multiple drugs comprises: (a) identifying a set of therapeutics being administered to the subject; (b) identifying excipients and APIs in the set of therapeutics being administered to the subject; (c) identifying an excipient being administered to the subject as the ARAII in the subject; (d) quantifying the total amount of the ARAII being administered to the subject to determine a first excipient burden; and (e) selecting a new set of therapeutics wherein the new set of therapeutics comprises the APIs of the first set of therapeutics and wherein the new set of therapeutics comprises a second excipient burden that is less than the first excipient burden.

In a thirteenth aspect, the invention relates to a method of designing a therapeutic formulation. In some embodiments, the method entails identifying a first therapeutic formulation comprising a first API and one or more excipients, wherein the first therapeutic formulation has previously been administered to a human.

In some embodiments, the first therapeutic formulation may be or may have been commercially available. In some embodiments the one or more excipients do not comprise an ARAII.

In some embodiments, the method further entails identifying a second API that is structurally similar to the first API.

In some embodiments, the second API has previously been administered to a human in at least one therapeutic formulation comprising an ARAII.

In some embodiments, the method further entails combining the one or more excipients of the first therapeutic formulation with the second API to arrive at a second therapeutic formulation.

In some embodiments, the second therapeutic formulation is not commercially available.

In a preferred embodiment of the thirteenth aspect of the invention, the method of designing a therapeutic formulation comprises: (a) identifying a first therapeutic formulation comprising a first API and one or more excipients, wherein the first therapeutic formulation has previously been administered to a human; (b) identifying a second API that is structurally similar to the first API; and (c) combining the one or more excipients of the first therapeutic formulation with the second API to arrive at a second therapeutic formulation.

An exemplary advantage of this aspect of the invention is that it provides an avenue for treating a subject with a specific API even if all known therapeutic formulations comprising that API also comprise an ARAII for the subject in need to treatment. Use of a formulation network, as described herein and exemplified in FIG. 7A, to organize and present interchangeabilities of therapeutic formulations for a number of different APIs may allow for the development of many new therapeutic formulations. Consequently, the invention may help to enable treatment of patients likely to suffer from exposure to certain ARAIIs by providing doctors and pharmacists with numerous therapeutic formulations to choose from for a given API.

Embodiments of the invention may serve to carefully align the precise mass of critical ingredients with the maximum dose tolerated by different patients to characterize affected patient populations and culprit formulations.

Further embodiments of the invention that account for effects of excipients may enable advanced formulations for difficult-to-deliver medications, allowing for the development of personalized medicine for vulnerable subpopulations. The methods disclosed empower clinicians to make conscious selections of formulations focusing on their patients' well-being and represent an advancement to the task of selecting an appropriate therapeutic for a given patient with potential implications for medical protocols, regulatory sciences, and pharmaceutical development of oral medications.

### Methods of Exploiting Underappreciated Activity of GRAS Compounds

While the machine learning methodologies of the disclosure will assist clinicians in identifying and replacing ARAIIs in therapeutic formulations, thereby reducing undesirable side effects among certain patient populations, these same methodologies also enable clinicians to identify heretofore unrecognized beneficial biological effects of generally-recognized-as-safe (GRAS) ingredients and compounds and to improve therapeutic formulations through the incorporation of these beneficial ingredients.

Accordingly, in yet another aspect, the invention relates to the use of machine learning to identify beneficial or adverse biological effects of GRAS ingredients and compounds or so-called inactive ingredients. In some embodiments, the invention relates to the identification of GRAS compounds or inactive ingredients capable of modulating the activity of enzymes, lysases, electrochemical transporters, GPCRs, or nuclear receptors. In some embodiments, the invention relates to the identification of GRAS ingredients and compounds or inactive ingredients capable of modulating the activity of enzymes, kinases, and family A GPCRs. In some embodiments, the invention relates to the identification of GRAS ingredients and compounds or inactive ingredients capable of modulating the activity of polyadenylate-biding protein 1, fatty acid-binding protein 3, sphingosine 1-phosphate receptor Edg-3, UGT2B7, or P-glycoprotein. In some embodiments, the invention relates to the identification of GRAS ingredients and compounds or inactive ingredients capable of modulating the activity of UGT2B7 or P-glycoprotein.

In another aspect, the invention relates to a method of improving liberation, absorption, distribution, metabolism, excretion, or toxicity (LADMET) of a therapeutic formulation. In an embodiment, the method comprises improving liberation. In an embodiment, the method comprises improving absorption. In an embodiment, the method comprises improving distribution. In an embodiment, the method comprises improving metabolism. In an embodiment, the method comprises improving excretion. In an embodiment, the method comprises improving toxicity.

In an embodiment, the method comprises (1) selecting an API and (2) formulating the API with a GRAS ingredient or compound, wherein the GRAS ingredient or compound is capable of modulating the activity of a metabolic protein or a transport protein, to produce a therapeutic formulation. In an embodiment the LADMET profile of the API is improved when formulated with the GRAS ingredient or compound with respect to the API when formulated without the GRAS ingredient or compound.

In some embodiments, the GRAS ingredient or compound is capable of modulating the activity of a metabolic protein. A metabolic protein may, for example, be an aminotransferase, a kinase, a member of the cytochrome P450 family, a glutathione S-transferase, a dehydrogenase, a sulfotransferase, an acyltransferase, or a glucuronosyltransferase. Nonlimiting examples of metabolic proteins include: CYP1A1, CYP1A2, CYP2A13, CYP2B6, CYP2C8, CYP2C9, CYP2D6, CYP2E1, CYP3A4, GSTP1, HK1, NAT2, NQO1, NT5C2, PCK1, SULT1A1, SULT2A1, TAT, and UGT2B7, Preferably, the metabolic protein is UGT2B7.

In some embodiments, the GRAS ingredient or compound is capable of modulating the activity of a transport protein. A transport protein may, for example, be a monocarboxylate transporter (MCT), multiple drug resistance protein (MDR), multidrug resistance-associated protein (MRP), peptide transporter (PEPT), or Na⁺ phosphate transporter (NPT). Nonlimiting examples of metabolic proteins include MCT1, MCT4, MRP2, P-glycoprotein, PEPT1, PEPT2, PHT1, and PHT2. Preferably, the transport protein is P-glycoprotein (P-gp), alternately known as multidrug resistance protein 1 (MDR1), ATP-binding cassette sub-gamily B member 1 (ABCB1), or cluster of differentiation 243 (CD243).

In some embodiments, the GRAS ingredient or compound is a UGT2B7 inhibitor. In some embodiments, the UGT2B7 inhibitor is selected from the group consisting of gum rosin or abietic acid. In some embodiments, the UGT2B7 inhibitor is gum rosin. In some embodiments, the UGT2B7 inhibitor is abietic acid. In some embodiments, the UGT2B7 inhibitor is not incorporated into the formulation as a coating.

In some embodiments, the GRAS ingredient or compound is a P-gp inhibitor. In some embodiments, the P-gp inhibitor is vitamin A palmitate.

In another aspect, the invention relates to a method of inhibiting UGT2B7 activity. In an embodiment, the method comprises contacting a cell having UGT2B7 activity with gum rosin or abietic acid. In an embodiment, the method comprises contacting a cell having UGT2B7 activity with gum rosin. In an embodiment, the method comprises contacting a cell having UGT2B7 activity with abietic acid. In an embodiment, the cell having UGT2B7 is a human cell.

In yet another aspect, the invention relates to a method of inhibiting P-gp activity. In an embodiment, the method comprises contacting a cell having P-gp activity with vitamin A palmitate. In an embodiment, the cell having P-gp activity is a human cell. In an embodiment, the cell having P-gp activity overexpresses P-gp. In an embodiment, the cell having P-gp activity is a cancer cell.

In another aspect, the invention relates to a method of treating a disease or disorder in a subject in need thereof comprising co-administering to the subject: (1) an effective amount of an active pharmaceutical ingredient (API), wherein the API undergoes UGT2B7-mediated glucuronidation and (2) a UGT2B7 inhibitor selected from the group consisting of gum rosin and abietic acid.

In an embodiment, the UGT2B7 inhibitor is gum rosin. In an embodiment, the UGT2B7 inhibitor is abietic acid.

In an embodiment, the API and the UGT2B7 inhibitor are co-administered as a formulation wherein the API and the UGT2B7 inhibitor are mixed together. Co-formulation as a mixture may, for example, entail blending together (e.g., in solution or in solid phase) the appropriate ratios of the API and the UGT2B7 inhibitor before molding into a pill or a capsule. In some embodiments, the API and the UGT2B7 inhibitor are co-formulated as a homogenous mixture. In an embodiment, the UGT2B7 inhibitor is not used as a coating.

Nonlimiting diseases and disorders that may be treated using the method include alcohol dependence, anxiety, benign prostatic hyperplasia, cancer (e.g., lung cancer, ovarian cancer, prostate cancer, leukemia, and breast cancer), chronic seizures, fever, high cholesterol, HIV infection, hypertension, inflammation, insomnia, lyperlipidemia, myocardial infarction, osteoarthritis, pain (moderate to severe), rheumatoid arthritis, stroke, and type 2 diabetes.

The API may, for example, be an opioid analgesic, an NSAID, an HMG-CoA reductase inhibitor, an adrenergic receptor agonist or antagonist, a benzodiazepine, an anticonvulsant, an SGLT2 inhibitor, or an estrogen receptor modulator.

By nonlimiting example, the API may be one of hydromorphone, losartan, diclofenac, etodolac, flurbiprofen, ibuprofen, naproxen, suprofen, mitiglinide, zaltoprofen, ambrisentan, troglitazone, morphine, indomethacin, mycophenolate mofetil, ezetimibe, mycophenolic acid, vadimezan, epirubicin, tapentadol, pitavastatin, silodosin, zidovudine, lovastatin, simvastatin, oxazepam, carbamazepine, codeine, fluvastatin, valproic acid, dapagliflozin, enasidenib, nalmefene, acemetacin, ertugliflozin, artenimol, labetalol, tamoxifen, carvedilol, ketorolac, dabigatran etexilate, dexibuprofen, gemfibrozil, anastrozole, or loxoprofen.

In an embodiment, UGT2B7-mediated glucuronidation is a primary metabolic pathway of the API.

In some embodiments, the API and the UGT2B7 inhibitor are co-formulated together. In some embodiments, the UGT2B7 is not formulated as a coating. In some embodiments, the API and the UGT2B7 inhibitor are administered at the same time. In some embodiments, the API and the UGT2B7 inhibitor are administered separately.

In some embodiments, the API has improved liberation, absorption, distribution, metabolism, excretion, or toxicity when co-administered with the UGT2B7 inhibitor when compared to administration of the API without the UGT2B7 inhibitor. In some embodiments, the API has improved metabolism when co-administered with the UGT2B7 inhibitor. In some embodiments, the API has improved excretion when co-administered with the UGT2B7 inhibitor. In some embodiments, the API has a longer half-life when co-administered with the UGT2B7 inhibitor. In some embodiments, the API has decreased clearance when co-administered with the UGT2B7 inhibitor.

In another aspect, the invention relates to a method of reducing the dose of a UGB2B7-sensitive API in a patient population being treated with the API comprising co-administering to the patient population: (1) a UGT2B7 inhibitor selected from the group consisting of gum rosin and abietic acid and (2) an effective amount of the API. In a preferred embodiment, the effective amount of the API being co-administered is lower than the effective amount required to induce the same therapeutic effect in the absence of the UGT2B7 inhibitor.

In an embodiment, the UGT2B7 inhibitor is gum rosin. In an embodiment, the UGT2B7 inhibitor is abietic acid. In an embodiment, the UGT2B7 inhibitor is not used as a coating.

In some embodiments, the patient population may be diagnosed, by nonlimiting example, with alcohol dependence, anxiety, benign prostatic hyperplasia, cancer (e.g., lung cancer, ovarian cancer, prostate cancer, leukemia, and breast cancer), chronic seizures, fever, high cholesterol, HIV infection, hypertension, inflammation, insomnia, lyperlipidemia, myocardial infarction, osteoarthritis, pain (moderate to severe), rheumatoid arthritis, stroke, or type 2 diabetes

In some embodiments, the UGB2B7-sensitive API may be, by nonlimiting example, an opioid analgesic, an NSAID, an HMG-CoA reductase inhibitor, an adrenergic receptor agonist or antagonist, a benzodiazepine, an anticonvulsant, an SGLT2 inhibitor, or an estrogen receptor modulator.

In some embodiments, the UBG2B7-sensitive API may be, by nonlimiting example, hydromorphone, losartan, diclofenac, etodolac, flurbiprofen, ibuprofen, naproxen, suprofen, mitiglinide, zaltoprofen, ambrisentan, troglitazone, morphine, indomethacin, mycophenolate mofetil, ezetimibe, mycophenolic acid, vadimezan, epirubicin, tapentadol, pitavastatin, silodosin, zidovudine, lovastatin, simvastatin, oxazepam, carbamazepine, codeine, fluvastatin, valproic acid, dapagliflozin, enasidenib, nalmefene, acemetacin, ertugliflozin, artenimol, labetalol, tamoxifen, carvedilol, ketorolac, dabigatran etexilate, dexibuprofen, gemfibrozil, anastrozole, or loxoprofen.

In some embodiments, the API and the UGT2B7 inhibitor are co-formulated together. In some embodiments, the UGT2B7 is not formulated as a coating. In some embodiments, the API and the UGT2B7 inhibitor are administered at the same time. In some embodiments, the API and the UGT2B7 inhibitor are administered separately.

In still another aspect, the invention relates to a method of treating a disease or disorder in a subject in need thereof comprising co-administering to the subject: (1) an effective amount of an active pharmaceutical ingredient (API), wherein the API is a P-gp substrate and (2) a P-gp inhibitor.

The P-gp inhibitor may, by nonlimiting example, be cholesterol, stearic acid, vitamin E, beta carotene, glyceril palmitate, or vitamin A palmitate. In a most preferable embodiment, the P-gp inhibtor is vitamin A palmitate.

The API may, by nonlimiting example, be irinotecan, ranitidine, colchicine, loperamide, or warfarin.

In an embodiment, co-administration of the API with the P-gp inhibitor results in increased cell permeability of the API. In an embodiment, co-administration of the API with the P-gp inhibitor results in increased oral absorption of the API. In an embodiment, co-administration of the API and the P-gp inhibitor results in increased oral absorption of the API by at least 35%, at least 30%, at least, 25%, at least 20%, at least 15%, at least 10%, or at least 5% with respect to administration of the API without the P-gp inhibitor.

More preferably, the invention relates to a method of treating cancer in a subject in need thereof comprising co-administering to the subject: (1) an effective amount of one or more chemotherapeutic agents and (2) a P-gp inhibitor.

The P-gp inhibitor may, by nonlimiting example, be cholesterol, stearic acid, vitamin E, beta carotene, glyceril palmitate, or vitamin A palmitate. In a most preferable embodiment, the P-gp inhibtor is vitamin A palmitate.

Accordingly, in a preferable aspect, the invention relates to a method of treating cancer in a subject in need thereof comprising co-administering to the subject: (1) an effective amount of one or more chemotherapeutic agents and (2) vitamin A palmitate.

In a preferred embodiment, the one or more chemotherapeutic agents is a P-gp substrate.

In an embodiment, vitamin A palmitate is co-administered in a therapeutically effective amount.

In an embodiment, the cancer is characterized by P-glycoprotein overexpression. In another embodiment, the cancer is multidrug-resistant cancer.

The term “cancer” refers to a disease or disorder caused by the proliferation of malignant neoplastic cells, such as tumors, neoplasms, carcinomas, sarcomas, leukemias, lymphomas and the like. For example, cancers include, but are not limited to, colorectal cancer, stomach cancer, pancreatic cancer, breast cancer, lung cancer, Non-Hodgkin's lymphoma, and ovarian cancer.

The chemotherapeutic agent may, for example, be an alkylating agent, a tumor necrosis factor, an intercalator, a microtubulin inhibitor, a topisomerase inhibitor, or a tyrosine kinase inhibitor.

Nonlimiting examples of chemotherapeutic agents compatible with the method of treating cancer include adriamycin, anastrozole, arsenic trioxide, asparaginase, azacytidine, BCG Live, bevacizumab, bexarotene capsules, bexarotene gel, bleomycin, bortezombi, busulfan intravenous, busulfan oral, calusterone, campothecin, capecitabine, carboplatin, carmustine, carmustine with polifeprosan 20 implant, celecoxib, cetuximab, chlorambucil, cisplatin, cladribine, clofarabine, cyclophosphamide, cytarabine, cytoxan, cytarabine liposomal, dacarbazine, dactinomycin, actinomycin D, dalteparin sodium, darbepoetin alfa, dasatinib, daunorubicin liposomal, daunorubicin, daunomycin, decitabine, denileukin, denileukin diftitox, dexrazoxane, dexrazoxane, docetaxel, doxorubicin, doxorubicin liposomal, dromostanolone propionate, eculizumab, Elliott's B Solution, epirubicin, epirubicin hcl, epoetin alfa, erlotinib, estramustine, etoposide phosphate, etoposide VP-16, exemestane, fentanyl citrate, filgrastim, floxuridine (intraarterial), fludarabine, fluorouracil 5-FU, fulvestrant, gefitinib, gemcitabine, gemcitabine hcl, gemicitabine, gemtuzumab ozogamicin, goserelin acetate, goserelin acetate, histrelin acetate, hydroxyurea, ibritumomab tiuxetan, idarubicin, ifosfamide, imatinib mesylate, interferon alfa 2a, interferon alfa-2b, irinotecan, lapatinib ditosylate, lenalidomide, letrozole, leucovorin, leuprolide acetate, levamisole, lomustine CCNU, meclorethamine, nitrogen mustard, megestrol acetate, melphalan L-PAM, mercaptopurine 6-MP, mesna, methotrexate, methoxsalen, mitomycin C, mitotane, mitoxantrone, nandrolone phenpropionate, nelarabine, nofetumomab, oprelvekin, oxaliplatin, paclitaxel, paclitaxel protein-bound particles, palifermin, pamidronate, panitumumab, pegademase, pegaspargase, pegfilgrastim, peginterferon alfa-2b, pemetrexed disodium, pentostatin, pipobroman, plicamycin, mithramycin, porfimer sodium, procarbazine, quinacrine, rasburicase, rituximab, sargramostim, sorafenib, streptozocin, sunitinib, sunitinib maleate, talc, tamoxifen, temozolomide, teniposide VM-26, testolactone, thalidomide, thioguanine 6-TG, thiotepa, topotecan, topotecan hcl, toremifene, tositumomab, tositumomab/I-131 tositumomab, trastuzumab, tretinoin ATRA, uracil mustard, valrubicin, vinblastine, vincristine, vinorelbine, vorinostat, zoledronate, zoledronic acid, and mixtures thereof.

In some embodiments, the chemotherapeutic agent and vitamin A palmitate are co-formulated together. In some embodiments, the chemotherapeutic agent and vitamin A palmitate are administered at the same time. In some embodiments, the chemotherapeutic agent and vitamin A palmitate are administered separately.

In some embodiments, the chemotherapeutic agent has improved liberation, absorption, distribution, metabolism, excretion, or toxicity when co-administered with vitamin A palmitate when compared to administration of the chemotherapeutic agent without vitamin A palmitate. In some embodiments, the chemotherapeutic agent has improved absorption when co- administered with vitamin A palmitate. In some embodiments, the chemotherapeutic agent has improved metabolism when co-administered with vitamin A palmitate. In an embodiment, co- administration of the chemotherapeutic agent with vitamin A palmitate results in increased cell permeability of the chemotherapeutic agent. In an embodiment, co-administration of the chemotherapeutic agent with vitamin A palmitate results in increased oral absorption of the chemotherapeutic agent. In an embodiment, co-administration of the chemotherapeutic agent and vitamin A palmitate results in increased oral absorption of the chemotherapeutic agent by at least 35%, at least 30%, at least, 25%, at least 20%, at least 15%, at least 10%, or at least 5% with respect to administration of the chemotherapeutic agent without vitamin A palmitate.

### Pharmaceutical Compositions

The invention further relates to pharmaceutical compositions.

In one aspect, the invention relates to a pharmaceutical composition comprising: (1) an active pharmaceutical composition (API), wherein the API undergoes UBT2B7-mediated glucuronidation and (2) a UGT2B7 inhibitor selected from the group consisting of gum rosin and abietic acid.

In some embodiments, the UGT2B7 inhibitor is gum rosin. In some embodiments, the UGT2B7 inhibitor is abietic acid.

In some embodiments, the API and the UGT2B7 inhibitor are co-formulated as a mixture. Co-formulation as a mixture may, for example, entail blending together (e.g., in solution or in solid phase) the appropriate ratios of the API and the UGT2B7 inhibitor before molding into a pill or a capsule. In some embodiments, the API and the UGT2B7 inhibitor are co-formulated as a homogenous mixture. In some embodiments, the UGT2B7 inhibitor is not used as a coating.

In another aspect, the invention relates to a pharmaceutical composition comprising a chemotherapeutic agent and vitamin A palmitate.

In a preferred embodiment, the chemotherapeutic agent is a P-gp substrate.

In further aspects, the invention relates to a pharmaceutical composition comprising vitamin A palmitate and an API selected from the group consisting of: irinotecan, ranitidine, colchicine, loperamide, and warfarin. In an embodiment, the API is irinotecan. In an embodiment, the API is ranitidine. In an embodiment, the API is colchicine. In an embodiment, the API is loperamide. In an embodiment, the API is warfarin.

### EXAMPLES

### Example 1: Inactive ingredients: A Major Component of Drug Mass

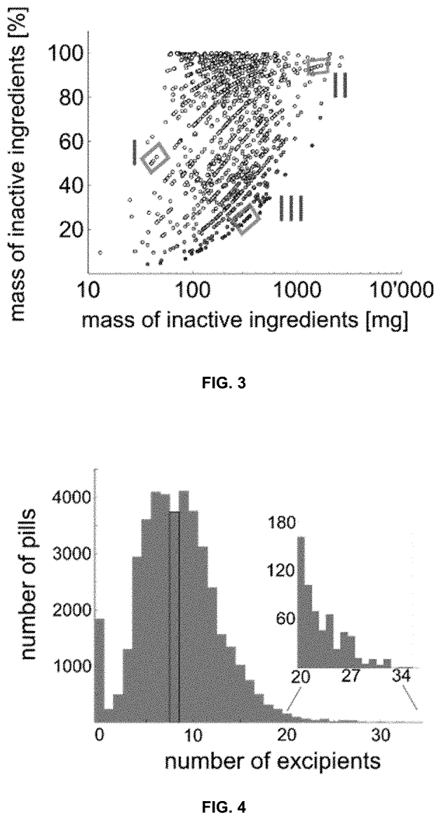

Oral solid dosage formulations of the most frequently prescribed medications in the United States as supplied from the pharmacy at Brigham and Women's Hospital consist of 75%±26% inactive ingredients (Table 1). The lipid-lowering agent atorvastatin calcium 80 mg (Major Pharma) is indicated for the prevention of various cardiovascular diseases and contains the largest amount of inactive ingredient among these pills, with an inactive ingredient mass of 770 mg. Simvastatin 5 mg (McKesson), belonging to the same medication class as atorvastatin, contained the lowest amount of inactive ingredients (50 mg), which nevertheless accounted for 90% of its total mass. The German database “Gelbe Liste” (www.gelbe-liste.de) captures the piece weights for a large set of 1,902 different medications, extending the scope of our analysis of the most frequently prescribed medication. We mined these data and observed a similar average value of 71%±26% (FIG. 2), highlighting that inactive ingredients make up the major part of the administered material. In terms of mass, an average tablet or capsule contains 280 mg of inactive ingredient and only 164 mg of API. Close to half (41.3%) of all drug products contain more than 250 mg of inactive ingredients (FIG. 3). Such doses are further multiplied by polypharmacy (simultaneous usage of multiple medications), which is particularly prevalent in older adults: 39.0% of Americans over the age of 65 take at least five prescription medications daily, and 11.7% of a similar cohort of Swedes took more than 10 prescription medications daily. A patient taking 10 prescription medications would ingest an average of 2.8 g of inactive ingredients daily. This is a substantial amount of excipient material that is administered to patients every day and merits further consideration.

TABLE 1
Piece weight analysis of different versions of most
commonly prescribed medications.

| | | | | Weight | Weight | Weight | % |
|---|---|---|---|---|---|---|---|
| Active ingredient | Dose | | Producer | 1 | 2 | 3 | Inactive |
| amlodipine | 2.5 | mg | Major Pharma | 49.3 | 49.0 | 48.9 | 92.93% |
| | 5 | mg | AvKARE/ | 202.1 | 202.0 | 202.2 | 96.57% |
| | | | AvPAK | | | | |
| | 10 | mg | McKesson | 203.9 | 203.7 | 203.6 | 93.19% |
| amoxicillin | 250 | mg | NorthStar Rx | 379.2 | 379.0 | 379.1 | 34.05% |
| | 500 | mg | NorthStar Rx | 717.0 | 717.3 | 716.5 | 30.26% |
| Atorvastatin | 20 | mg | AvKARE/ | 206.4 | 206.0 | 206.1 | 89.50% |
| | | | AvPAK | | | | |
| | 80 | mg | Major Pharma | 856.3 | 857.0 | 856.7 | 89.89% |
| azithromycin | 250 | mg | American | 463.3 | 463.2 | 462.8 | 44.72% |
| | | | Health | | | | |
| | | | Packaging | | | | |
| | 600 | mg | Teva | 1052.4 | 1052.3 | 1052.3 | 41.61% |
| furosemide | 20 | mg | West Ward | 85.3 | 85.5 | 85.4 | 76.58% |
| | 40 | mg | West Ward | 168.6 | 168.2 | 168.6 | 76.26% |
| | 80 | mg | West Ward | 339.8 | 339.7 | 339.6 | 76.45% |
| gabapentin | 100 | mg | McKesson | 177.2 | 176.8 | 177.2 | 43.52% |
| | 300 | mg | McKesson | 462.3 | 462.4 | 462.3 | 35.11% |
| | 400 | mg | McKesson | 610.1 | 610.3 | 609.8 | 34.43% |
| hydrochlorothiazide | 25 | mg | McKesson | 110.5 | 110.7 | 110.5 | 77.39% |
| ibuprofen | 200 | mg | LNK | 327.1 | 326.6 | 326.8 | 38.81% |
| | 400 | mg | McKesson | 567.3 | 566.8 | 567.2 | 29.47% |
| | 600 | mg | McKesson | 867.7 | 867.9 | 867.9 | 30.86% |
| | 800 | mg | McKesson | 1179.8 | 1179.4 | 1179.6 | 32.18% |
| levothyroxine | 25 | mcg | Mylan Inst | 130.2 | 129.7 | 130.0 | 99.98% |
| | 50 | mcg | Mylan Inst | 129.0 | 128.8 | 128.9 | 99.96% |
| | 75 | mcg | Mylan Inst | 130.1 | 129.6 | 130.0 | 99.94% |
| | 88 | mcg | Mylan Inst | 131.2 | 130.8 | 131.0 | 99.93% |
| | 100 | mcg | Mylan Inst | 128.6 | 128.7 | 128.4 | 99.92% |
| | 112 | mcg | Mylan Inst | 130.6 | 130.5 | 130.2 | 99.91% |
| | 125 | mcg | Mylan Inst | 129.8 | 129.5 | 129.6 | 99.90% |
| | 125 | mcg | AbbVie | 128.9 | 130.2 | 129.6 | 99.90% |
| | 125 | mcg | AbbVie | 131.1 | 131.2 | 130.7 | 99.90% |
| | 137 | mcg | Mylan Inst | 128.8 | 128.8 | 129.2 | 99.89% |
| | 150 | mcg | Mylan Inst | 130.1 | 130.0 | 129.9 | 99.88% |
| | 200 | mcg | AbbVie | 132.3 | 131.9 | 131.7 | 99.85% |
| lisinopril | 2.5 | mg | Qualitest | 97.0 | 96.9 | 96.6 | 97.42% |
| | 5 | mg | Major Pharma | 106.9 | 106.8 | 106.7 | 95.32% |
| | 10 | mg | Major Pharma | 213.6 | 213.9 | 213.6 | 95.32% |
| | 20 | mg | McKesson | 194.9 | 194.6 | 194.7 | 89.73% |
| | 20 | mg | McKesson | 195.8 | 195.9 | 196.4 | 89.80% |
| losartan | 25 | mg | McKesson | 92.3 | 92.4 | 92.2 | 72.91% |
| | 50 | mg | McKesson | 182.9 | 183.2 | 182.8 | 72.67% |
| metformin | 500 | mg | McKesson | 602.6 | 602.1 | 602.5 | 17.00% |
| | 850 | mg | Major Pharma | 961.6 | 961.6 | 961.8 | 11.61% |
| metoprolol tartrate | 25 | mg | American | 146.9 | 146.7 | 146.3 | 83.75% |
| | | | Health | | | | |
| | | | Packaging | | | | |
| | 50 | mg | American | 291.7 | 292.0 | 291.7 | 83.67% |
| | | | Health | | | | |
| | | | Packaging | | | | |
| | 50 | mg | American | 292.8 | 292.2 | 292.9 | 83.71% |
| | | | Health | | | | |
| | | | Packaging | | | | |
| | 100 | mg | American | 482.6 | 484.4 | 482.3 | 80.27% |
| | | | Health | | | | |
| | | | Packaging | | | | |
| metoprolol | 25 | mg | Major Pharma | 102.3 | 102.3 | 102.0 | 75.54% |
| succinate | 50 | mg | Major Pharma | 205.5 | 205.1 | 205.9 | 75.67% |
| omeprazole | 20 | mg | Major Pharma | 309.9 | 310.1 | 309.7 | 93.55% |
| sertraline | 25 | mg | American | 78.2 | 78.1 | 77.6 | 64.16% |
| | | | Health | | | | |
| | | | Packaging | | | | |
| | 50 | mg | American | 153.8 | 153.7 | 153.4 | 63.63% |
| | | | Health | | | | |
| | | | Packaging | | | | |
| | 100 | mg | American | 307.6 | 307.7 | 307.8 | 63.68% |
| | | | Health | | | | |
| | | | Packaging | | | | |
| simvastatin | 5 | mg | McKesson | 49.9 | 49.9 | 50.0 | 89.99% |
| | 10 | mg | McKesson | 100.8 | 100.8 | 100.3 | 90.06% |
| | 20 | mg | McKesson | 204.2 | 203.9 | 204.2 | 90.20% |

### Example 2: Complexity of the Formulation Landscape

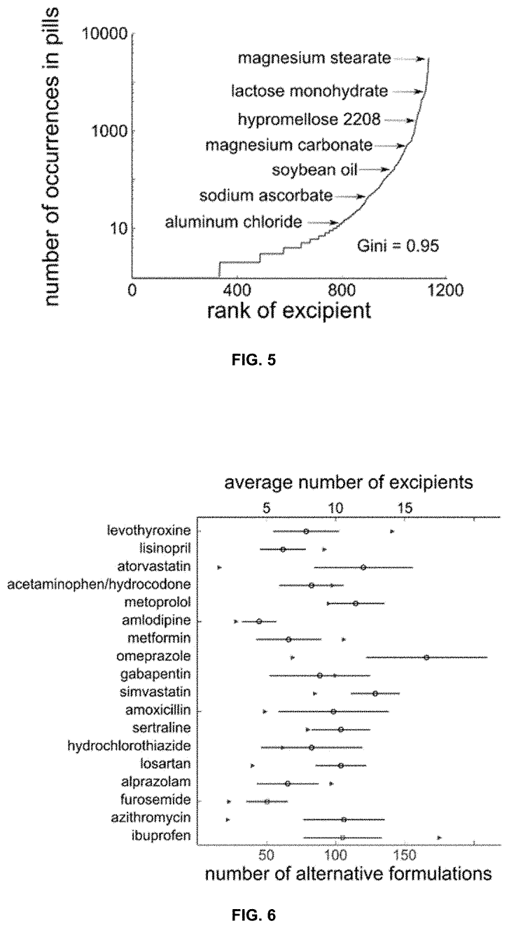

The Pillbox database (https://pillbox.nlm.nih.gov) contains information on 42,052 oral solid dosage formulations consisting of a total of 354,597 inactive ingredients. According to this data, an average tablet or capsule contains 8.8 inactive ingredients (FIG. 4). 596 oral solid dosage forms contain 20 different inactive ingredients or more. Individual inactive ingredients occur in vastly different numbers (FIG. 5, Table 2): magnesium stearate can be found in 30,263 oral solid dosage forms (72%), whereas a third of all inactive ingredients (333, 30%) only occur once. We calculated the Gini index to measure disparity in inactive ingredient occurrence. The Gini index is a value ranging from zero (perfect equality, all ingredients occur in the same frequency) to one (perfect inequality, only a single ingredient occurs in all medications and other ingredients never occur). A Gini index of 0.95, close to perfect inequality, indicates that the number of occurrences of inactive ingredients is heavily skewed towards the most commonly occurring inactive ingredients.

TABLE 2
Top ten most common inactive ingredients in Pillbox.

| | Number of occurrences | |
|---|---|---|
| Inactive ingredient | in Pillbox (total 42,052) | |
| magnesium stearate | 30,263 | (72%) |
| microcrystalline cellulose | 23,325 | (55%) |
| titanium dioxide | 21,125 | (50%) |
| silicon dioxide | 15,612 | (37%) |
| starch corn | 15,405 | (37%) |
| lactose monohydrate | 11,658 | (28%) |
| hypromelloses | 11,547 | (27%) |
| talc | 10,472 | (25%) |
| croscarmel lose sodium | 8760 | (21%) |
| polyethylene glycols | 8282 | (20%) |

On average, 82.5 alternative formulations are available per API for the 18 most frequently prescribed oral medications in the US (FIG. 6), highlighting the multiplicity of available versions of the same medication. For example, 140 distinct formulations of the hypothyroidism treatment levothyroxine are produced by 43 different manufactures. Varying numbers of included inactive ingredients in such formulations indicates that different commercially-available versions of medications can contain different excipient mixtures. A “formulation network” can visualize this relationship on a larger scale, depicting available alternatives of all oral solid dosage forms and interchangeabilities of inactive ingredients (FIG. 7A). The network consists of a total of 13,287 nodes, corresponding to the number of unique combinations of inactive ingredients available in Pillbox. The network is populated with a total of 314,866 edges that highlight interchangeability of formulations. Only 1,003 formulations (7.5%) appear unique (isolated nodes on the periphery of the network). Most of these (668, 67%) have been reported only for a single API. A much larger fraction of the network corresponds to inactive ingredient combinations that have been used interchangeably for at least one API (mean value 3.12). These nodes build a convoluted network with distinct relationships between the formulations, highlighting the complexity of available alternatives. A mean degree of 23.7 indicates that, on average, more than 23 alternative combinations of inactive ingredients have been commercialized to deliver the same APIs. These results highlight the multiplicity of available alternatives of medications in terms of their inactive ingredient portion and warrants further study of the differences between those alternatives.

### Example 3: Adverse Reactions Associated with Excipients

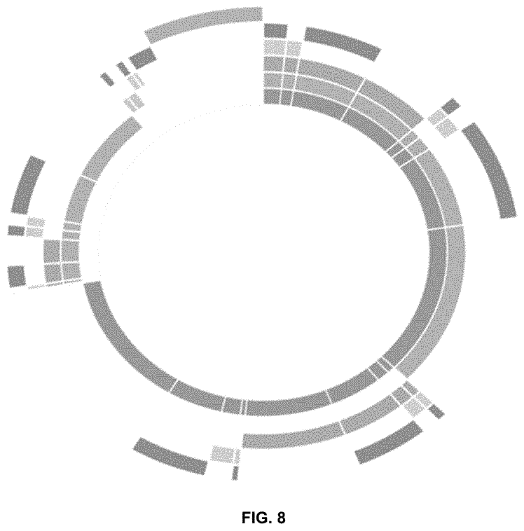

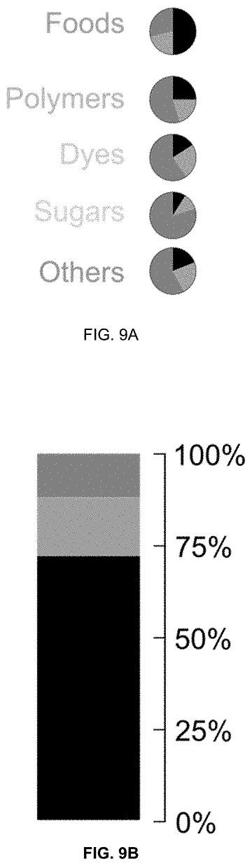

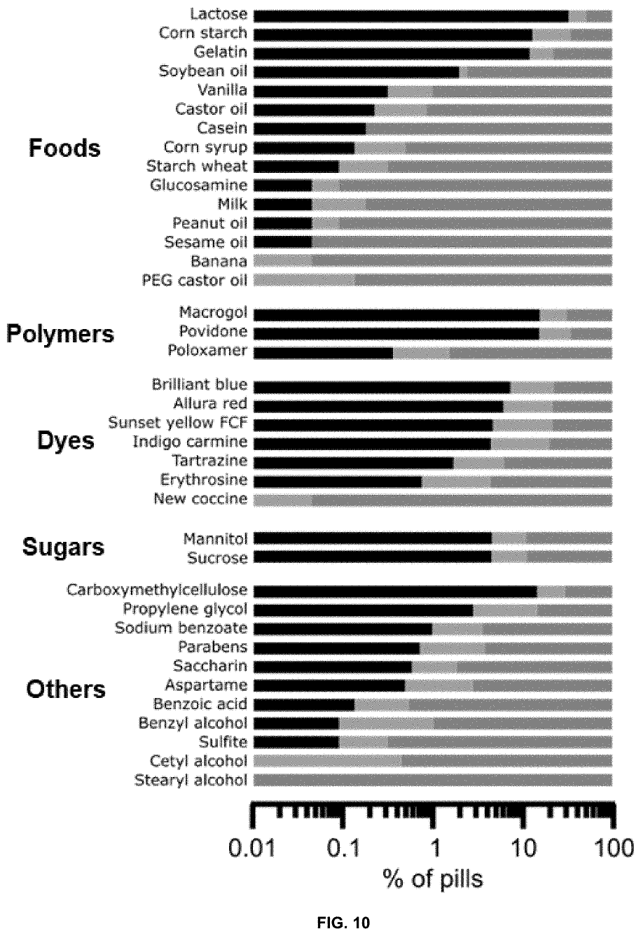

A total of 38 inactive ingredients (Table 3) have been described to cause allergic symptoms after oral exposure. (Table 4) These associations are supported by re-challenge with the isolated ARAII or the report of the patient tolerating an alternative formulation. Although these inactive ingredients occur in widely different frequencies, a Gini index of 0.75 is lower for ARAIIs compared to all inactive ingredients—indicating a more homogeneous occurrence among medications. Almost all oral solids (92.8%) contain at least one potential allergen (FIG. 8). Viewed through the lens of the APIs, only 28% of active ingredients have at least one available formulation that avoids all of these potential allergens, and only 12% of APIs are free of inactive ingredients that have been reported to cause allergic reactions (FIG. 9A and FIG. 9B). In many cases, particular APIs will contain a specific ARAII in all available formulations. For example, all available rosuvastatin calcium and diclofenac tablets, among others, contain lactose as an inactive ingredient (FIG. 10).

TABLE 3
List of critical inactive ingredients that can act
as allergens. Percentage occurrence refers
to fraction of all formulations of medications (solid
oral dosage forms) that contain the critical ingredient.

| | | Percentage occurrence |
|---|---|---|
| Ingredient | Classification | in medications |
| Lactose | food | 44.82% |
| Corn starch | food | 36.54% |
| PEG | polymer | 36.03% |
| Povidone | polymer | 35.80% |
| Carboxymethylcellulose | other | 21.38% |
| Gelatin | food | 16.93% |
| Brilliant blue | dye | 14.47% |
| Sunset Yellow FCF | dye | 12.27% |
| Allura red | dye | 11.20% |
| Propylene glycol | other | 11.14% |
| Indigo carmine | dye | 10.63% |
| Mannitol | sugar | 7.20% |
| Sucrose | sugar | 5.21% |
| Sodium benzoate | other | 1.72% |
| Parabens | other | 1.48% |
| Aspartame | other | 1.46% |
| Erythrosine | dye | 1.03% |
| Tartrazine | dye | 0.95% |
| Saccharin | other | 0.81% |
| Poloxamer | polymer | 0.76% |
| Soybean oil | food | 0.44% |
| Benzyl alcohol | other | 0.43% |
| Vanilla | food | 0.38% |
| Castor oil | food | 0.30% |
| Cetyl alcohol | other | 0.19% |
| Sulfite | other | 0.19% |
| PEG castor oils | food | 0.13% |
| Peanut oil | food | 0.08% |
| Benzoic acid | other | 0.07% |
| Corn syrup | food | 0.05% |
| Sesame Oil | food | 0.05% |
| Starch wheat | food | 0.04% |
| Casein | food | 0.03% |
| Banana essence | food | 0.01% |
| Milk | food | 0.01% |
| Glucosamine | food | 0.00% |
| New coccine | dye | 0.00% |
| Stearyl alcohol | other | 0.00% |

TABLE 4
List of publications analyzed for identification
of reports of allergic reactions or gastrointestinal
side effects through inactive ingredients in medications.

| | | Extracted inactive ingredients |
|---|---|---|
| PMID | Included? | or reason to exclude |
| 28684647 | Yes | Parabens and benzoates |
| 28613520 | No | Not relevant |
| 28163222 | No | Not relevant |
| 27882527 | No | Not relevant |
| 27834127 | Yes | Carboxymethylcellulose (CMC), povidones, |
| | | PEG (macrogols), sulfites, benzyl alcohol, |
| | | and tweens |
| 27712572 | Yes | Polyethylene glycol (PEG) |
| 27534768 | No | Inhaled medications |
| 28827390 | Yes | Wheat starch, peanut oil/arachis oil, benzyl |
| | | alcohol |
| 27491381 | No | Not relevant |
| 27436328 | No | Not relevant |
| 27196817 | Yes | PEG, polysorbates (Tweens), poloxamers, |
| | | PEG castor oils, laureth-9, cetomacrogol, |
| | | PEG 40 stearate, cetomacrogol 1000, |
| | | PEG 6000, polysorbate 80, hydroxyethylated |
| | | starch, poloxamer, polysorbate 80 |
| 27128715 | No | Environmental |
| 26636421 | No | Not relevant |
| 26419538 | No | Topical medications |
| 26211812 | Yes | Succinate esters, carboxymethylcellulose |
| | | (CMC), polyethylene glycol |
| | | (PEG; macrogol), lactose |
| 26156542 | No | Topical medications |
| 25885102 | No | Topical medications |
| 25764151 | No | Inhaled medications |
| 25751935 | No | Injections |
| 25514481 | No | Nasal and respiratory delivery only |
| 25384223 | No | Topical drugs |
| 25341165 | No | Injections |
| 25017684 | Yes | Carboxymethylcellulose (also called |
| | | carmellose or croscarmellose, sodium |
| | | carboxymethylcellulose, and E466), |
| | | tartrazine, FD&C Blue No. 1 (bright blue), |
| | | Blue No. 2 (indigo carmine), orange disperse 3 |
| | | (Sunset Yellow), Povidone (PVP, polyvinyl- |
| | | pyrrolidone), Sodium benzoate (E211), sulfites |
| 25017683 | No | Topical drugs |
| 24878443 | Yes | Gelatin, milk, casein, lactose, lactulose |
| 24832168 | No | Topical drugs |
| 24714850 | No | Injections |
| 24674688 | No | Injections |
| 24656778 | No | Topical drugs |
| 24565702 | No | Not relevant |
| 24559657 | No | Not relevant |
| 24456019 | No | Topical drugs |
| 24173385 | No | No adverse effects reported |
| 24051350 | No | Injections |
| 24002150 | No | Topical drugs |
| 23765411 | No | No inactive ingredients discussed |
| 23730887 | No | All IV or SC formulations |
| 23544966 | No | Inhaled medications |
| 23543606 | No | No adverse effects reported |
| 23504430 | No | Not relevant |
| 23340678 | No | Not relevant |
| 23339763 | No | Not relevant |
| 23292495 | No | Injections |
| 25674402 | No | Focus on delivery properties instead of |
| | | adverse reactions |
| 23243989 | No | Japanese article |
| 23238161 | No | Injections |
| 22833905 | No | No focus on adverse events |
| 22707362 | No | Focus on stability instead of adverse events |
| 22394125 | No | Topical drug |
| 22312932 | Yes | Casein, lactose, banana essence, vanilla, |
| | | vanillin |
| 24300191 | No | Ophthalmic products |
| 22099411 | No | French article |
| 21801484 | No | Not relevant |
| 21787819 | No | Nanomedicines |
| 21741802 | No | Treatment |
| 21626047 | No | Nutritional supplements |
| 21611683 | No | Topical |
| 21199198 | No | Inhaled medications |
| 20949699 | No | Subcutaneous injection |
| 20861601 | No | Parenteral |
| 20517534 | No | Parenteral |
| 20185893 | No | Topical drug |
| 20128230 | Yes | Carboxymethylcellulose (also called CMC, |
| | | carmelose) |
| 20013666 | No | Otic drops |
| 19732201 | Yes | Indigo carmine (E132), sunset yellow, |
| | | quinoline yellow |
| 19580371 | Yes | Corn syrup, benzalkonium chloride, allura |
| | | red (E129; FD&C Red No 40), brilliant |
| | | blue (E133; FD&C Blue No 1), erythrosine |
| | | (E127; FD&C Red No 3), indigo carmine |
| | | (E132; FD&C Blue No 2), Sunset yellow |
| | | (E110; FD&C Yellow No 6), tartrazine |
| | | (E102; FD&C Yellow No 5) |
| 19567843 | Yes | Methylhydroxybenzoate, propylhydroxy- |
| | | benzoate, cetyl alcohol, stearyl alcohol, |
| | | polysorbate 80, arachis oil |
| 19467048 | No | Topical dental |
| 19240542 | No | Topical/ocular drugs |
| 18845195 | No | Topical/ocular drugs |
| 18830864 | No | Generally topical |
| 18497245 | No | German, topical |
| 17159596 | No | Parenteral |
| 17037081 | No | Preserving transplants |
| 17017934 | No | Ophthalmic products |
| 16960822 | No | Focused on chemical reactions |
| 16868222 | No | Comparing opioid formulations |
| 16792601 | No | Discussion of allergen tolerance |
| 16572992 | No | Chinese |
| 16303277 | No | Discussion of terms, not the products |
| 16180936 | No | Discussion of terms, not the products |
| 16018907 | No | Ingredient discussed is only used in |
| | | parenterals; it isn't in pillbox |
| 15996453 | No | Parenteral |
| 15788144 | No | Topical sunscreens |
| 15778049 | No | Focused on chemical reactions |
| 15714807 | No | Chinese |
| 14977910 | No | Parenteral |
| 13679965 | No | Spanish |
| 12964493 | Yes | Amaranth, benzalkonium chloride, sunset |
| | | yellow, parabens, peanut oil, ponceau, |
| | | sulfites, tartrazine, brilliant black BN |
| | | (E151), carmoisine (E122, azorubine), |
| | | Bronopol, Castor Oil, Corn starch, |
| | | mercury, Sesame Oil, Soybean oil |
| 12871181 | Yes | Carboxymethylcellulose |
| 12721396 | No | Parenteral, focus on actives |
| 12614517 | No | Topical, focus on microbicides |
| 12042063 | No | Topical/ocular drugs |
| 11392448 | No | Parenteral |
| 11392447 | No | Parenteral |
| 11361009 | No | Focused on chemical reactions |
| 11325479 | No | Focused on chemical reactions |
| 11135703 | No | Parenteral |
| 10502611 | No | Focused on pharmaceutical properties |
| 10229638 | No | Focused on breakdown of protein/peptide |
| | | products |
| 9057785 | Yes | Diethylene glycol |
| 9024461 | Yes | Sulfur dioxide, sodium sulfite, sodium |
| | | bisulfite, potassium bisulfite, sodium |
| | | metabisulfite, and potassium |
| | | metabisulfite, Aspartame, Saccharin, |
| | | tartrazine (FD&C Yellow No. 5), |
| | | sunset yellow, new coccine, amaranth, |
| | | erythrosine, indigo carmine (FD&C Blue |
| | | No. 2), ponceau, Brilliant Blue (FD&C |
| | | Blue No. 1), methyl blue, quinolone |
| | | yellow, FD&C Red No. 40, lactose, |
| | | propylene glycol, Benzalkonium chloride, |
| | | Allura red (E129; FD&C Red No 40), |
| | | Brilliant blue (E133; FD&C Blue |
| | | No 1), Erythrosine (E127; FD&C Red |
| | | No 3), Indigo carmine (E132; FD&C |
| | | Blue No 2), Sunset yellow (E110; |
| | | FD&C Yellow No 6), Tartrazine (E102; |
| | | FD&C Yellow No 5) |
| 8877241 | Yes | FD&C yellow #5 ( tartrazine), FD&C |
| | | yellow #6 (sunset yellow), FD&C Blue |
| | | propylparaben, aspartame, mannitol, sucrose |
| 8766194 | No | Inhaled medications |
| 8644576 | No | Topical/ocular drugs |
| 8729891 | No | Parenteral |
| 8571282 | No | French |
| 7600718 | No | Topical sunscreens |
| 8535931 | Yes | Carboxymethylcellulose, sulfer dioxide, |
| | | Tartrazine (yellow dye No. 5, E 102), ponceau, |
| | | erythrosine, Benzoic acid, Aminobenzoic |
| | | acid, para-hydroxybenzoic acid (parabens) |
| 7551218 | No | Used for injected medications |
| 7842686 | No | Topical meds |
| 8378865 | No | German |
| 7912532 | No | Injections |
| 1421646 | No | Reaction to katerolac, an active ingredient |
| 1497796 | No | Parenteral/sc |

As opposed to the small number of patients who experience severe allergic reactions to inactive ingredients, many more patients are vulnerable to experiencing adverse symptoms caused by the inactive ingredients. For example, the symptoms of irritable bowel syndrome (IBS) are being increasingly managed in part by a diet that is low in fermentable oligosaccharides, disaccharides, monosaccharides, and polyols (FODMAPs). 55% of all oral medications contained at least one FODMAP sugar in their formulation, and 5% contained more than one FODMAP sugar. The most commonly occurring FODMAPs are lactose, mannitol, and polydextrose, found in 45%, 7%, and 4% of all oral solids, respectively. Quantities of these sugars could exceed 500 mg per pill (Table 5), contributing to increased FODMAP consumption and potential discomfort.

TABLE 5
Lactose content of various medications.

| | Lactose | |
|---|---|---|
| | content | Pubmed |
| Drug | [mg] | ID |
| Allegron 10 mg (Nortriptyline) | 38.00 | 19035974 |
| Allopurinol | 57.00-171.00 | 24732384 |
| Amitryptiline 10 mg | 43.00 | 19035974 |
| Amlodipine | 140.00-151.00 | 24732384 |
| Asacol MR 400 mg (Mesalazine) | 75.0 | 19035974 |
| Azathioprine | 34.36-116.00 | 24732384 |
| Bisoprolol | 1.26-136.00 | 24732384 |
| Budenofalk 3 mg (Budesonide) | 600.0 | 19035974 |
| Capecitabine | 7.00-68.95 | 24732384 |
| Celevac 500 mg (Methylcelluose) | 27.70 | 19035974 |
| Citalopram 20 mg | 45.00 | 19035974 |
| Clozapine | 32.44-281.62 | 24732384 |
| Codeine phosphate 30 mg | 46.00 | 19035974 |
| Colofac 135 mg (Mebeverine HCl) | 95.00 | 19035974 |
| Delta-cortil 5 mg (Prednisolone) | 31.0 | 19035974 |
| Destolit 150 mg (Ursodeoxycholic acid) | 78.00 | 19035974 |
| Domperidone 10 mg | 56.00 | 19035974 |
| Du!co-Lax 5 mg (Bisacodyl) | 41.00 | 19035974 |
| Enalapril | 78.00-253.60 | 24732384 |
| Fluconazole | 16.60-210.00 | 24732384 |
| Imodium 2 mg (Loperamide HCl) | 108.00 | 19035974 |
| Imodium 2 mg (Loperamide HCl) | 125.00 | 19035974 |
| Imuran 50 mg (Azathioprine) | 71.0 | 19035974 |
| Levofloxacin | 3.60-26.45 | 24732384 |
| Loratadine | 62.50-75.00 | 24732384 |
| Losartan | 4.50-231.60 | 24732384 |
| Losec 40 mg (Omeprazole) | 4.00 | 19035974 |
| Mebeverine hydrochloride 135 mg | 99.00 | 19035974 |
| Merbentyl 10 mg (Dicycloverine HCl) | 74.00 | 19035974 |
| Mesren MR 400 mg (Mesalazine) | 77.0 | 19035974 |
| Methotrexate 2.5 mg (Methotrexate) | 28.9 | 19035974 |
| Metoclopramide 10 mg | 71.00 | 19035974 |
| Morphine 10 mg | 90.00 | 21766071 |
| Morphine 30 mg | 70.00 | 21766071 |
| Nevirapine | 168.00-464.00 | 24732384 |
| OxyContin 10 mg (Oxycodone) | 69.25 | 21766071 |
| OxyContin 20 mg (Oxycodone) | 59.25 | 21766071 |
| OxyContin 40 mg (Oxycodone) | 32.25 | 21766071 |
| OxyContin 80 mg (Oxycodone) | 78.50 | 21766071 |
| Pancrex V tablets (Pancreatin) | 54.00 | 19035974 |
| Prednisolone 2.5 mg | 56.0 | 19035974 |
| Pro-banthine 15 mg (Propantheline Br) | 38.00 | 19035974 |
| Prochlorperazine 5 mg | 70.00 | 19035974 |
| Puri-Nethol 50 mg (Mercaptopurine) | 61.0 | 19035974 |
| Senokot 7.5 mg (Senna) | 16.00 | 19035974 |
| Simvastatin | 35.00-576.24 | 24732384 |
| Zoton Fastab 30 mg (Lansoprazole) | 28.00 | 19035974 |

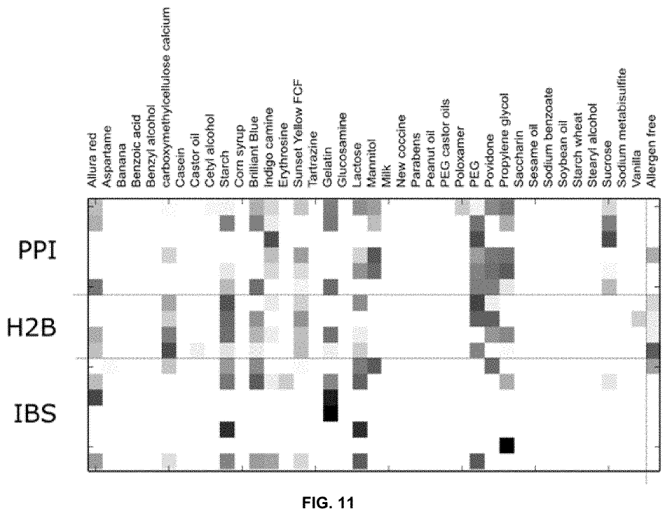

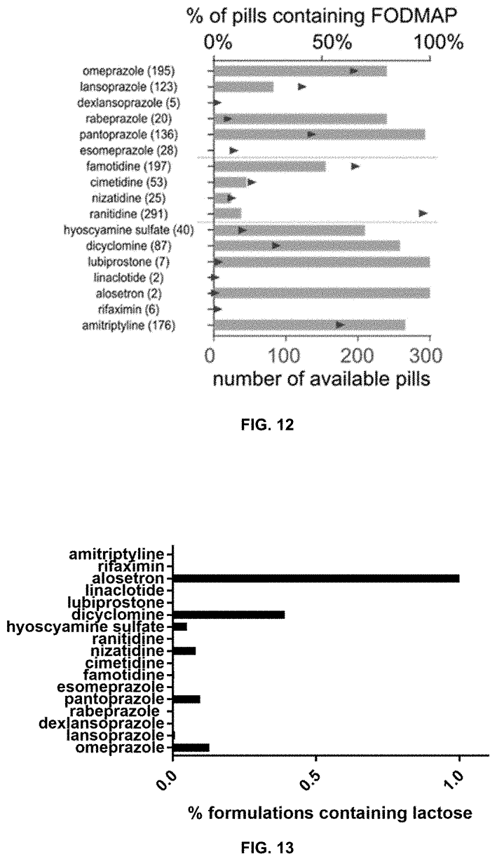

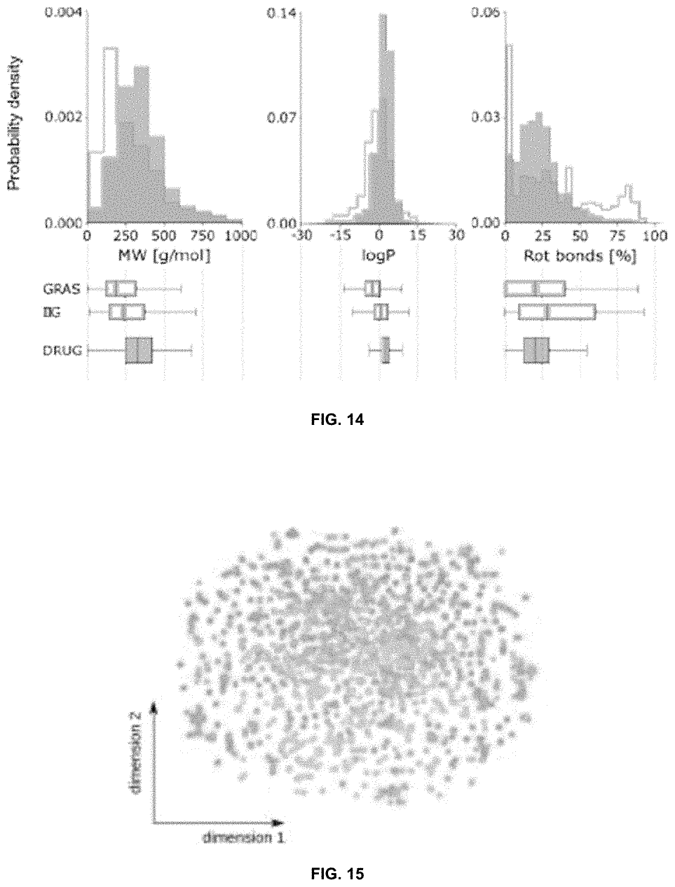

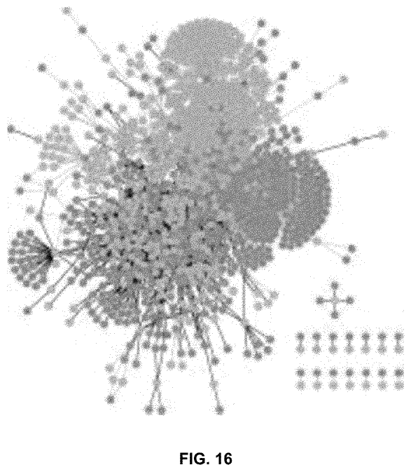

Allergen ARAII and FODMAP content in oral medications to manage gastrointestinal symptoms is of particular concern because recipients of these medications may experience a worsening of their symptoms due to these ingredients. Certain medication classes are more likely to contain specific ARAIIs, although there were often available medications in the same class that avoided those inactive ingredients (FIG. 11). For example, polymers such as povidone, polyethylene glycol (PEG), and propylene glycol occur commonly in proton pump inhibitors (PPI), with the exception of dexlansoprazole. Rifaximin tablets (used for treating IBS) contain propylene glycol, which might worsen symptoms. We found that FODMAP sugars were commonly included in formulations across gastrointestinal drug classes, but every investigated class had FODMAP-free alternatives (FIG. 12). These data highlight the need for appropriate selection of not only the API but also the formulation as a whole to help mitigate adverse reactions or improve symptom control in some patients.

### Example 4: Lactose, Peanut Oil, Gluten, and Chemical Dyes

In addition to lactose's role as an allergen and FODMAP sugar, lactose intolerance is present in 75% of the world population. Nevertheless, lactose is commonly used in 45% of all oral solid dosage forms (Table 1), with lactose content reaching up to 600 mg per pill (Table 5). Lactose intake from medications has been associated with adverse reactions in multiple published case reports, although whether low quantities of lactose elicit reactions remains debated. It appears lactose content in medications is too small to cause symptoms for many patients, but individuals with severe cases of lactose intolerance could be affected by less than 200 mg of lactose, an amount possibly exceeded by a single medication (Table 5). Furthermore, patients with multiple comorbidities could be more susceptible given their exposure to multiple medications each day: for instance, a patient with hypertension and high cholesterol could be on a regimen of amlodipine, simvastatin, and losartan with a combined daily load of lactose close to 1 g (Table 5). Under-recognition of the lactose content in medications could be an avoidable cause of medication non-compliance or discontinuation that could be mistakenly attributed to the API.

Conversely, allergens can cause severe reactions even at a very low exposure, with lowest-observed-adverse-effect levels (LOAEL) in the sub milligram range, which might trigger reactions after administering only a single agent. For many such ingredients, manufacturers include warning labels emphasizing the physiological relevance of this association. For example, according to the Pillbox data, 100% of progesterone and 62.5% of valproic acid capsules contain peanut oil as a solubilizer (FIG. 10). APIs with such formulations cannot be taken by patients with peanut allergies, prohibiting therapeutic opportunities. Estimates on the prevalence of peanut allergy reach up to 4% of the US population with a growing incidence in children. Some formulations of valproic acid replace peanut oil with corn oil, supporting the potential to confer safer adverse effect profiles by substituting critical ingredients with possibly more benign alternatives (FIG. 7B).

Gluten can cause severe reactions in patients suffering from celiac disease at doses as low as 1.5 mg daily when exposed chronically. Inactive ingredients produced from wheat starch can result in gluten content in medications. In a survey, 18% of manufacturers indicated that their medications contain gluten. Although 69% claimed to produce gluten-free products, only 17% tested their products and could provide documentation on the performed tests. The FDA has recently recommended adding gluten content to product labels, indicating an increasing awareness of the potential risk for patients.

Chemical dyes, such as tartrazine, have been suspected to cause severe atopic reactions, specifically in patients with existing allergic or asthmatic conditions. However, 33% of all medications contain at least one chemical dye associated with allergic reactions in patients (FIG. 8, Table 1). Researchers have conducted trails to investigate allergic reactions in patients receiving tartrazine-containing medications versus the same patients receiving tartrazine-free alternatives. These trials observed adverse symptoms associated with tartrazine content in about 4% of all patients and higher incidence in sensitive subgroups.

These data support the potential of inactive ingredients as the cause of adverse events in patients.

### Example 5: Machine Learning Predicts Novel Biological Associations of Inactive Ingredients and GRAS Compounds

### Datasets

Generally-recognized-as-safe (GRAS) compounds and inactive ingredients were retrieved from the FDA website as CAS codes. The codes were converted into SMILES structures using the NIH CACTUS server and subsequently manually curated. The DrugBank database (version 5.0) was extracted in XML format and post-processed in Python to extract all SMILES strings for small molecules in the category “approved.” ChEMBL22 served as the reference database for bioactive compounds to enable machine learning-based predictions. ChEMBL22 bioactivities (IC₅₀, Kᵢ, EC₅₀) were logarithmized into pAffinity values. In accordance with standard protocol, Kᵢ values were shifted, inconclusive data was removed, and multiple activity entries were averaged as long as their standard-deviation was below one, and activities annotated as lower bounds were penalized by one logarithmic unit.

### Machine Learning Predictions

Structures of GRAS compounds and inactive ingredients, as well as known bioactive compounds from ChEMBL22 were encoded using Morgan fingerprints (radius 4, 2048 bits) as well as physicochemical properties using the RDkit (Landrum, 2012) in Python. These descriptors were used to build Random Forest regression models in scikit-learn using 500 trees and considering all features for every tree. The model was retrospectively evaluated using ten-fold cross validation with shuffling for every investigated protein. For prioritization of predictions, the predicted pAffinity of the GRAS compounds or inactive ingredients was considered. These predictions were normalized based on a random set of compounds extracted from ChemDB that we had subsampled to ensure an equivalent molecular weight distribution compared to the safe compound libraries through Probability Proportional to Size (PPS) Sampling. This generated standardized prediction scores that were used to rank computational predictions for selection of downstream validation.

### Biological Associations

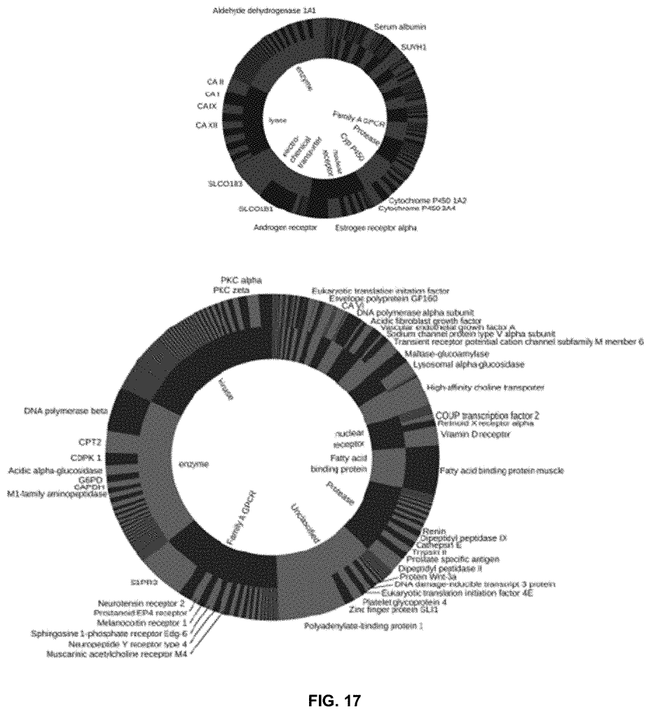

Restricting predictions only to those whose pAffinity exceeded 1.5 standard deviations of the mean prediction for the background dataset, a total of 1907 predicted ligand-target associations for GRAS compounds and inactive ingredients were identified, two-fold more than currently known activities for these molecules (FIG. 17). The three most frequently predicted targets for GRAS compounds and inactive ingredients were polyadenylate-binding protein 1 (127 predictions), fatty acid-binding protein 3 (95 predictions), and sphingosine 1-phosphate receptor Edg-3 (89 predictions), which are implicated in oculopharyngeal muscular dystrophy, cardiac fatty acid utilization, and multiple sclerosis, respectively. Overall, the three most commonly predicted protein classes were enzymes (343 predictions), kinases (343 predictions), and family A GPCRs (280 predictions), supporting the unmapped potential of GRAS compounds and inactive ingredients to exert adverse reactions through biological effects, act as starting points for drug discovery projects, or enhance treatments as functional supplements. Importantly, there was no correlation between the number of previously measured bioactivities and the number of predicted bioactivities of a GRAS compound/inactive ingredient, signifying that there is a vast uncharted poly-pharmacological space of safe compounds and that the disclosed machine learning approach acts independently from previously acquired biological activity data for GRAS compounds and inactive ingredients.

### Example 6: Gum Rosin and Abietic Acid Inhibit UGT2B7 In Vitro and Ex Vivo

### UGT2B7 Inhibition Assay

UGT2B7 inhibition was measured using Corning® Supersomes™ Human UGT2B7. The inhibition of UGT2B7 was measured using the commercially-available Biovision UGT activity screening kit as previously described. Briefly, 0.1 mg/mL microsomes were mixed with Alamethicin for pore-formation and a proprietary UGT ligand that loses fluorescence after glucuronidation (Biovision). Plates were incubated for 5 minutes at RT and protected from light before the enzymatic reaction was initiated through the addition of UDPGA. Loss of fluorescence was measured after 30 minutes on a microplate reader (Infinite M200, Tecan) and compared to the loss of fluorescence in the presence of different concentrations of gum rosin or abietic acid dissolved in PBS with 1% DMSO. Diclofenac (1 mM in PBS 1% DMSO) served as a positive inhibitor control.

### UGT Tissue Assay

Compound mixtures were prepared at 500 μM. Freshly extracted porcine liver was homogenized using a tissue homogenizer. The sample was separated using centrifugation and the supernatant was used as a test sample. Two independent experiments with two different liver extracts were performed as described for the microsomes.

### Computational Docking

A homology model of human UGT2B7 was created using the SwissModel server based on the amino acid sequence of UGT2B7 as stored in Uniprot (UniProt ID P16662). The top-scoring homology model was based on a crystal structure for UGT85H2 (PDB ID 2pq6.1) and was used for docking in SwissDock. The molecular structure of abietic acid was provided via its ZINC ID (ZINC2267806). The highest scored binding mode with an estimated ΔG of −7.79 kcal/mol was extracted using UCSF Chimera and visualized in PyMol.

### In Vitro and Ex Vitro Activity of Gum Rosin and Abietic Acid

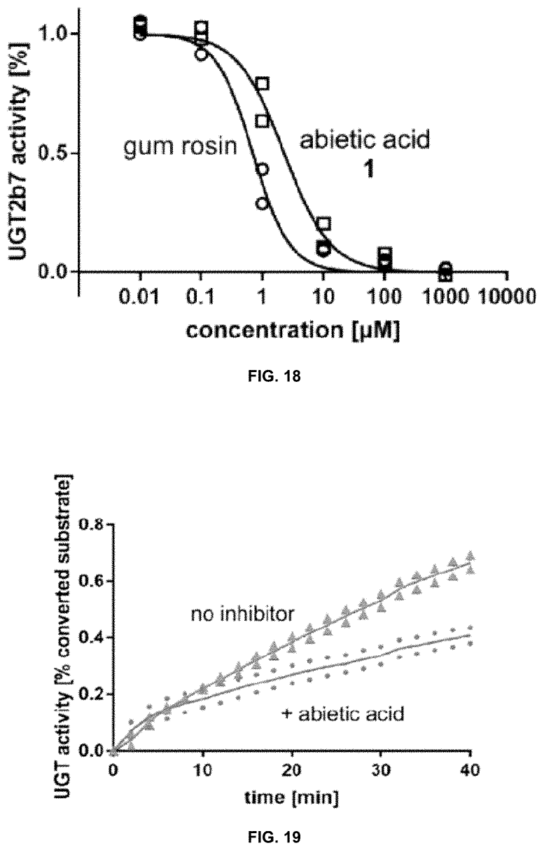

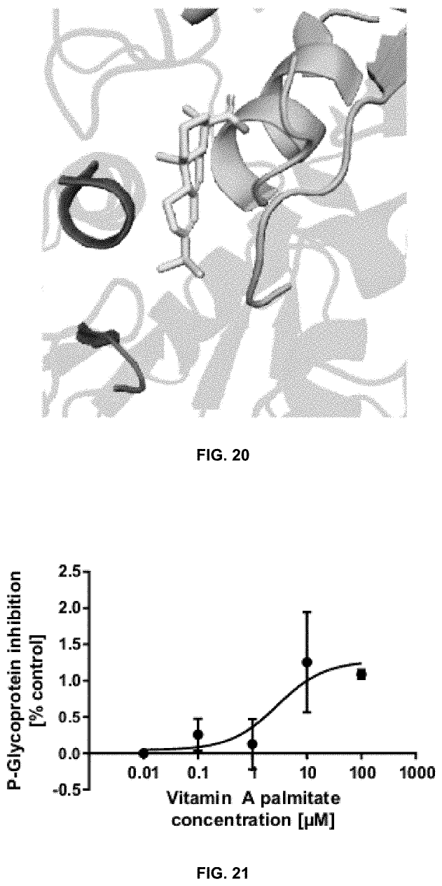

A machine learning approach was employed of identify safe inhibitors of glucuronidation through UGT2B7, a major metabolic pathway that affects about 10% of all drugs. Many drugs and toxins have been reported as UGT2B7 inhibitors, recognized through drug-drug interactions leading to significant changes in drug exposure and altering treatment efficiency and toxicity. Machine learning suggested gum rosin as the most promising safe inhibitor of UGT2B7 with an estimated IC₅₀ value of 2.8 μM based on the chemical structure of its main component abietic acid. Gum rosin is an FDA inactive ingredient and is used as a glazing agent in pills and chewing gums with E number E915. According to Pillbox data, rosin is currently included in pills of Rifater and ChlorTrimenton 12 Hour. Gum rosin's main component, abietic acid, is among the most soluble and least toxic resin acids and is harmless in mice. The most similar training compound with known UGT2B7 activity was isolongifolic acid (IC₅₀=2 μM). The machine learning model predicted that these distinct compounds would exhibit a similar pharmacophoric interaction pattern and provide an equivalent inhibition of UGT2B7 activity. Indeed, abietic acid inhibited the activity of UGT2B7 in a functional in vitro assay with an IC₅₀ value of 2.2±0.3 μM (FIG. 18). Unpurified gum rosin exhibited a slightly improved IC₅₀ value of 0.8±0.1 μM in the same in vitro assay, suggesting that abietic is a major but potentially not the only ingredient of gum rosin to inhibit UGT2B7 activity (FIG. 18). To confirm these effects in a more complex ex vivo environment, abietic acid was screened in a UGT tissue assay using pig liver lysate. Abietic acid successfully inhibited UGT activity and slowed the conversion of UGT substrates in the ex vivo assay (FIG. 19). To determine the potential mode of interaction of abietic acid with UGT2B7, molecular docking of abietic acid in a homology model of UGT2B7 was performed. The most probably binding mode identified positions abietic acid at the interface between the catalytic site and the co-factor binding domain, thereby potentially disrupting the interaction of the co-factor uridine diphosphate glucuronic acid with the substrates (FIG. 20).

### Example 7: Vitamin A Palmitate Inhibits P-glycoprotein Activity

### P-gp Inhibition Assay

HepG2 cells were used as model cells with MDR1 expression. Cells were plated at 40,000 cells per well in 200 μL DMEM+10% FBS+1% pen-strep. Cells were incubated overnight in 5% CO₂ atmosphere at 37° C. Cells were then washed and incubated with different concentrations of vitamin A palmitate in 1% DMSO PBS or 100 μM verapamil as the positive control. A proprietary, fluorogenic P-gp substrate (Biovision) was added and the sample was protected from light and incubated at 37° C. in a 5% CO₂ atmosphere. Fluorescence of the substrate (excitation 488 nm, emission 532 nm) was measured after 12 hours.

### P-gp Tissue Assay

Four P-gp substrates (irinotecan, ranitidine, colchicine, and loperamide) were prepared in a 5% DMSO PBS solution at concentrations of 1 mg/mL, while vitamin A palmitate was prepared in the same solution with a final working concentration of 400 μM. Fresh porcine intestinal tissue was washed according to previously published protocols. A high-throughput “intestine on a chip” screening system was setup to determine substrate permeability on tissue incubated with the vitamin A palmitate solution for 30 minutes compared to tissue that was incubated with 5% DMSO PBS for the same duration as buffer control. After 60 minutes of permeability, irinotecan was detected using UV-VIS fluorescence (excitation 370, emission 470), and ranitidine, colchicine, and loperamide were detected using absorption at 312 nm, 350 nm, and 415 nm, respectively.

### P-gp In Vivo Experiment

A suspension of vitamin A palmitate in 10% DMSO PBS or 10% DMSO PBS buffer control were administered orally to five female BALB/c mice at a dose of 500 mg/kg 15 minutes prior to treatment. Mice were then treated orally with warfarin 20 mg/kg. Blood was sampled after 30 minutes of oral warfarin administration. Warfarin plasma concentrations were determined using LCMS.

### Computational Docking

The crustal structure of human P-glycoprotein was extracted from the PDB (PDB ID 6cov) and the cytosolic portion without any bound ATP was isolated in PyMol. UCSF Chimera was used for pre-processing of the structure using “dock prep” with default parameters. The molecular structure of vitamin A palmitate was extracted from PubChem and transformed into a MOL2 file in KNIME. Docking was performed on the SwissDock server. The top scoring binding mode with an estimated ΔG of -8.71 kcal/mol was extracted using UCSF Chimera and visualized in PyMol. For visualizing the ATPase domain, a mesh was created from atoms surrounding the co-crystalized ATP with a maximal distance of 5 Å.

### P-glycoprotein Inhibition

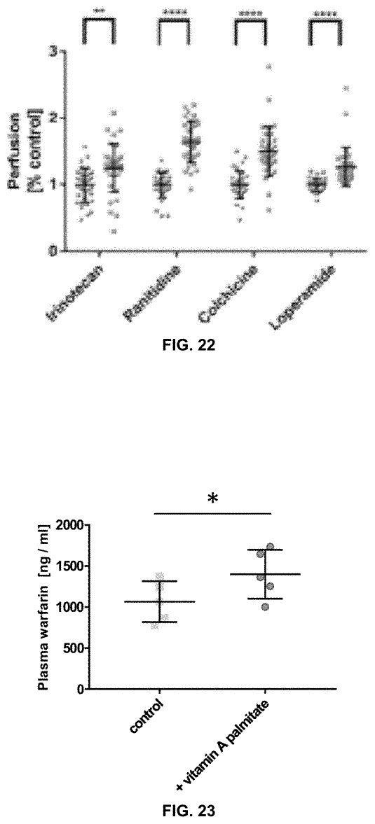

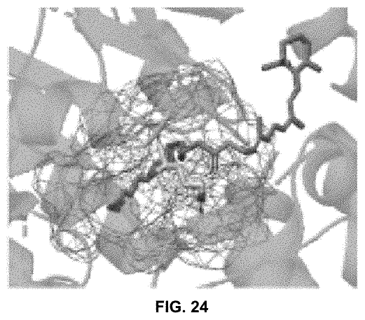

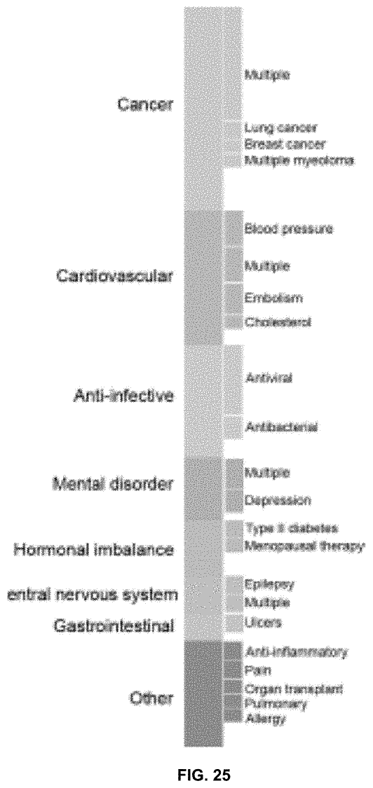

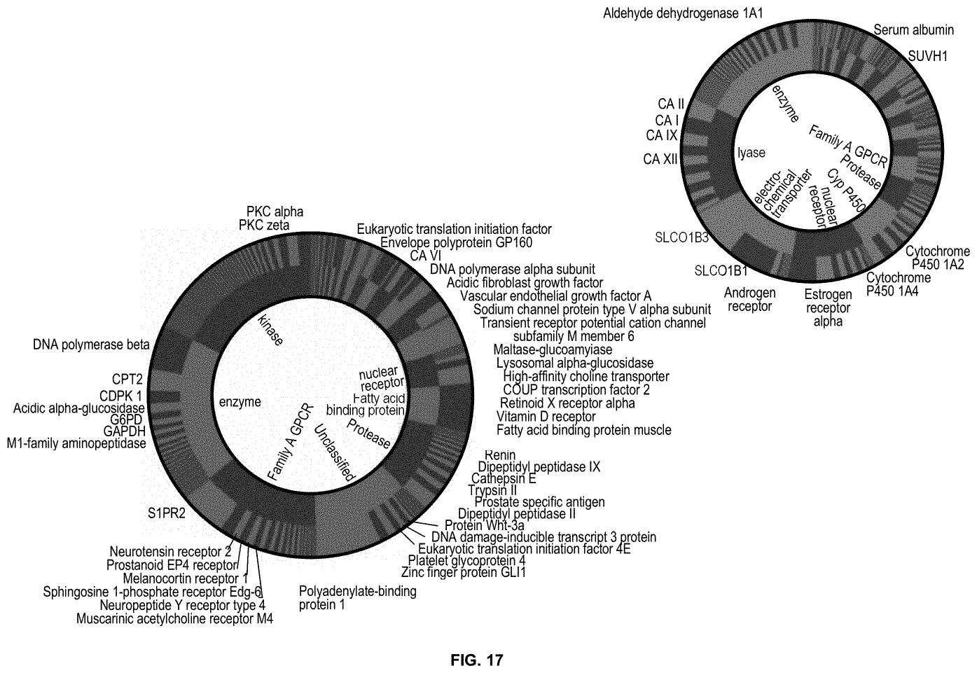

P-gp is one of the main active drug transporters and modulation of its activity can drastically impact the pharmacokinetics of 8% of currently approved therapeutics spanning various important disease areas (FIG. 25). Using machine learning, one of the highest scoring and novel predictions of P-gp inhibition was made for vitamin A palmitate. This prediction was confirmed in a cell-based in vitro assay, where vitamin A palmitate inhibited P-gp-mediated efflux of a fluorescent reporter substrate with an IC₅₀ value of 2.9±3.6 μM (FIG. 21). In an ex vivo Franz diffusion cell assay, vitamin A palmitate increased the permeability of irinotecan, ranitidine, colchicine, and loperamide—four FDA approved drugs that are known P-gp substrates (FIG. 22). Further, vitamin A palmitate increased the oral absorption of warfarin in mice by 31% (FIG. 23). Molecular docking studies indicate that this effect might be caused by the palmitate tail occupying the ATPase site, stabilized by an additional hydrogen bond involving the P-gp arginine residue at position 1047 (FIG. 24). This effect could constitute an important food-drug interaction and be harnessed in formulation development for drugs with transport liabilities.

### REFERENCES

1. A. Katdare, M. V. Chaubal, Eds., Excipient Development for Pharmaceutical, Biotechnology, and Drug Delivery Systems (Informa Healthcare, 2006).

2. C. G. Abrantes, D. Duarte, C. P. Reis, An Overview of Pharmaceutical Excipients: Safe or Not Safe?, J. Pharm. Sci. 105, 2019-2026 (2016).

3. M. P. Jensen, P. Karoly, Handbook of Pharmaceutical Excipients—7th Edition (The Pharmaceutical Press, 2013).

4. D. P. Elder, M. Kuentz, Pharmaceutical excipients—quality, regulatory and biopharmaceutical considerations, Eur. J. Pharm. Sci. 87, 88-99 (2016).

5. J. M. Kelso, Potential food allergens in medications, J. Allergy Clin. Immunol. 133, 1509-1520 (2014).

6. R. A. Swerlick, C. F. Campbell, Medication dyes as a source of drug allergy., J. drugs dermatology JDD 12, 99-102 (2013).

7. R. D. Brandstetter, R. Conetta, B. Glazer, Lactose Intolerance Associated with Intal Capsules, New Engl. J. Med. 315, 1613-1614 (1986).

8. FDA, Gluten in Drug Products and Associated Labeling Recommendations, Draft Guid. (2017).

9. S. Salunke, F. Liu, H. Batchelor, J. Walsh, R. Turner, T. R. Ju, C. Tuleu, European Paediatric Formulation Initiative (EuPFI)—Formulating Ideas for Better Medicines for Children, AAPS PharmSciTech 18, 257-262 (2017).

10. Drug allergy: An updated practice parameter. Ann. Allergy, Asthma Immunol. 105, 259-273 (2010).

11. M. S. Bhatia, Allergy to tartrazine in psychotropic drugs, J. Clin. Psychiatr. 61, 473-476 (2000).

12. E. P. Halmos, V. A. Power, S. J. Shepherd, P. R. Gibson, J. G. Muir, A Diet Low in FODMAPs Reduces Symptoms of Irritable Bowel Syndrome, Gastroenterology 146, 67-75.e5 (2014).

13. D. Mill, J. Dawson, J. L. Johnson, Managing acute pain in patients who report lactose intolerance: the safety of an old excipient re-examined, Ther. Adv. Drug Saf. (2018), doi:10.1177/2042098617751498.

14. M. Aitken, M. Kleinrock, Medicines Use and Spending in the U.S.: A Review of 2016 and Outlook to 2021., Rep. by QuintilesIMS Institute. (2017).

15. C. J. Charlesworth, E. Smit, D. S. H. Lee, F. Alramadhan, M. C. Odden, Polypharmacy Among Adults Aged 65 Years and Older in the United States: 1988-2010., J Gerontol A Biol Sci Med Sci 70, 989-95 (2015).

16. L. Morin, K. Johnell, M.-L. Laroche, J. Fastbom, J. W. Wastesson, The epidemiology of polypharmacy in older adults: register-based prospective cohort study., Clin. Epidemiol. 10, 289-298 (2018).

17. F. L. Suarez, D. A. Savaiano, M. D. Levitt, A Comparison of Symptoms after the Consumption of Milk or Lactose-Hydrolyzed Milk by People with Self-Reported Severe Lactose Intolerance, New Engl. J. Med. 333, 1-4 (1995).

18. J. Lieb, D. J. Kazienko, Lactose Filler as a Cause of Drug-Induced Diarrhea, New Engl. J. Med. 299, 314-314 (1978).

19. L. Petrini, P. Usai, A. Caradonna, R. Cabula, S. Mariotti, Lactose intolerance following antithyroid drug medications, J. Endocrinol. Investig. 20, 569-570 (1997).

20. M. Guslandi, Lactose content of gastrointestinal drugs: does it matter?, Aliment. Pharmacol. Ther. 29, 1212-1212 (2009).

21. M. Montalto, A. Gallo, L. Santoro, F. D'Onofrio, V. Curigiliano, M. Covino, G. Cammarota, A. Grieco, A. Gasbarrini, G. Gasbarrini, Low-dose lactose in drugs neither increases breath hydrogen excretion nor causes gastrointestinal symptoms, Aliment. Pharmacol. Ther. 28, 1003-1012 (2008).

22. FDA, Approaches to establish thresholds for major food allergens and for gluten in food. J. Food Prot., 108 (2006).

23. A. L. Cohen, L. Neumayer, K. Boucher, R. E. Factor, G. Shrestha, M. Wade, J. G. Lamb, K. Arbogast, S. R. Piccolo, J. Riegert, M. Schabel, A. H. Bild, T. L. Werner, Window-of-Opportunity Study of Valproic Acid in Breast Cancer Testing a Gene Expression Biomarker, JCO Precis. Oncol., 1-11 (2017).

24. A. W. Burks, Peanut allergy, Lancet 371, 1538-1546 (2008).

25. K. M. Nagel-Edwards, J. Y. Ko, Excipient choices for special populations, Int. J. Pharm. Compd. 12, 426-430 (2008).

26. J. Reid, A. Kelly, S. McDonald, Systematic review of safe level of gluten for people with coeliac disease (Cochrane Australia, 2016).

27. L. J. Chartrand, P. A. Russo, A. G. Duhame, E. G. Seidman, Wheat Starch Intolerance in Patients With Celiac Disease, J. Am. Diet. Assoc. 97, 612-618 (1997).

28. A. R. King, Gluten Content of the Top 200 Medications: Follow-Up to the Influence of Gluten on a Patient's Medication Choices., Hosp. Pharm. 48, 736-43 (2013).

29. B. S. M. Stenius, M. Lemola, Hypersensitivity to acetylsalicylic acid (ASA) and tartrazine in patients with asthma, Clin. Exp. Allergy 6, 119-129 (1976).

30. I. Neuman, R. Elian, H. Nahum, P. Shaked, D. Creter, The danger of “yellow dyes” (tartrazine) to allergic subjects, Clin. Exp. Allergy 8, 65-68 (1978).

31. M. E. MacCara, Tartrazine: a potentially hazardous dye in Canadian drugs., Can. Med. Assoc. J. 126, 910-4 (1982).

32. D. Amaral Silva, R. Löbenberg, N. Davies, Are Excipients Inert? Phenytoin Pharmaceutical Investigations with New Incompatibility Insights., J Pharm Pharm Sci 21, 29745 (2018).

33. W. Zhang, Y. Li, P. Zou, M. Wu, Z. Zhang, T. Zhang, The Effects of Pharmaceutical Excipients on Gastrointestinal Tract Metabolic Enzymes and Transporters—an Update, APPS J. 18, 830-843 (2016).

1. A method of selecting a therapeutic for an individual subject, the method comprising the steps of:
a) providing a formulation network that depicts available alternatives of dosage forms and interchangeabilities of functionally equivalent inactive ingredients,
b) selecting a first drug formulation from within the formulation network wherein the first drug formulation comprises an active pharmaceutical ingredient and at least one inactive ingredient,
c) identifying at least one ingredient that is toxic to the individual subject from among the at least one inactive ingredient in the first drug formulation, and
d) selecting and administering a second drug formulation from within the formulation network wherein the second drug formulation comprises the active pharmaceutical ingredient and does not comprise the at least one toxic ingredient.

2. A method of treating a subject with a therapeutic comprising an active pharmaceutical ingredient (API), the method comprising
a) evaluating a first set of excipients provided with an API for toxicity in the subject;
b) replacing the first set of excipients wherein one or more of the excipients is found to be toxic to the subject with a second set of excipients, wherein the toxic excipients in the first set of excipients are replaced with non-toxic excipients that are functionally equivalent to the corresponding toxic excipient; and
c) administering the API with the second set of excipients to the subject.

3. The method of claim 2, wherein the toxicity is an allergy.

4. The method of claim 3, wherein the allergy is to gluten or lactose.

5. The method of claim 2, wherein the functional equivalence is selected from the group consisting of antiadherence, binding, coating, color, disintegration, flavor, providing glide, lubrication, preservation of the API, prevention of water absorption, sweetening, bulking, vehicles, and bioequivalents.

6. The method of claim 2, wherein the one or more excipients found to be toxic to the subject are selected from the group consisting of a food, a polymer, a dye, and a sugar.

7. The method of claim 2, wherein the one or more excipients found to be toxic to the subject are selected from the group consisting of lactose, corn starch, PEG, povidone, carboxymethylcellulose, gelatin, Brilliant Blue, Sunset Yellow FCF, Allura Red, propylene glycol, indigo carmine, mannitol, sucrose, sodium benzoate, parabens, aspartame, erythrosine, tartrazine, saccharine, poloxamer, soybean oil, benzyl alcohol, vanilla, castor oil, cetyl alcohol, sulfite, PEG castor oils, peanut oil, benzoic acid, corn syrup, sesame oil, starch wheat, casein, banana essence, milk, glucosamine, new coccine, and stearyl alcohol.

8. The method of claim 2, wherein the toxicity is gastrointestinal distress.

9. The method of claim 8, wherein the gastrointestinal distress is caused by a fermentable oligosaccharide, disaccharide, monosaccharide, or polyol (FODMAP).

10. The method of claim 2, wherein the method further comprises mitigating toxicity in the subject, wherein the toxicity is the result of at least one toxic inactive ingredient in the first set of excipients.

11. The method of claim 2, wherein the subject has an allergy;
the toxic excipient in the first set of excipients is an allergen to the subject;
and the second set of excipients does not comprise the allergen.

12. (canceled)

13. The method of claim 1, wherein the subject is ingesting multiple drugs, and wherein the method further comprises comprising:
a) identifying all excipients in the multiple drugs being ingested by the subject; and
b) quantifying the total amount of each excipient being ingested by the subject during a specified timeframe to determine an excipient burden.

14. The method of claim 13, further comprising:
c) identifying one or more symptoms experienced by the subject during administration of the multiple drugs, and
d) correlating the excipient burden to the one or more symptoms to establish a potential causal relationship.

15. The method of claim 2, wherein the subject has irritable bowel syndrome, small intestinal bacterial overgrowth, or dyspepsia;
the toxic excipient in the first set of excipients provokes an adverse reaction in the subject's gastrointestinal (GI) tract; and
the second set of excipients comprises a reduced amount of the toxic excipient.

16-24. (canceled)

25. A method of reducing the total adverse reaction-associated inactive ingredient (ARAII) excipient burden in a subject being administered multiple drugs, the method comprising:
a) identifying a set of therapeutics being administered to the subject;
b) identifying excipients and APIs in the set of therapeutics being administered to the subject;
c) identifying an excipient being administered to the subject as the ARAII in the subject;
d) quantifying the total amount of the ARAII being administered to the subject to determine a first ARAII excipient burden; and
e) selecting a new set of therapeutics wherein the new set of therapeutics comprises the APIs of the first set of therapeutics and wherein the new set of therapeutics comprises a second ARAII excipient burden that is less than the first ARAII excipient burden.

26-31. (canceled)

32. The method of claim 2, wherein the first set of excipients and the second set of excipients have previously been administered to a human.

33. (canceled)

34. A method of inhibiting UGT2B7 activity, comprising contacting a cell having UGT2B7 activity with a UGT2B7 inhibitor selected from the group consisting of gum rosin and abietic acid.

35. (canceled)

36. A method of treating a disease or disorder in a subject in need thereof comprising co-administering to the subject:
an effective amount of an active pharmaceutical ingredient (API), wherein the API undergoes UGT2B7-mediated glucuronidation; and
a UGT2B7 inhibitor selected from the group consisting of gum rosin and abietic acid.

37. The method of claim 36, wherein the UGT2B7 inhibitor and the API are co-administered in a formulation wherein the UGT2B7 inhibitor and the API are mixed together.

38. The method of claim 36, wherein the UGT2B7 inhibitor is not used as a coating.

39. The method of claim 36, wherein the API is selected from the group consisting of: hydromorphone, losartan, diclofenac, etodolac, flurbiprofen, ibuprofen, naproxen, suprofen, mitiglinide, zaltoprofen, ambrisentan, troglitazone, morphine, indomethacin, mycophenolate mofetil, ezetimibe, mycophenolic acid, vadimezan, epirubicin, tapentadol, pitavastatin, silodosin, zidovudine, lovastatin, simvastatin, oxazepam, carbamazepine, codeine, fluvastatin, valproic acid, dapagliflozin, enasidenib, nalmefene, acemetacin, ertugliflozin, artenimol, labetalol, tamoxifen, carvedilol, ketorolac, dabigatran etexilate, dexibuprofen, gemfibrozil, anastrozole, and loxoprofen.

40. (canceled)

41. A method of inhibiting P-glycoprotein activity, comprising contacting a cell having P-glycoprotein activity with vitamin A palmitate.

42. The method of claim 41, wherein the cell overexpresses P-glycoprotein.

43. A method of treating cancer in a subject in need thereof comprising co-administering to the subject:
an effective amount of one or more chemotherapeutic agents; and
vitamin A palmitate.

44. The method of claim 43, wherein the cancer is characterized by P-glycoprotein overexpression.

45. The method of claim 43, wherein the cancer is multidrug-resistant cancer.

46. The method of claim 43, wherein the chemotherapeutic is selected from the group consisting of alkylating agents, tumor necrosis factors, intercalators, microtubulin inhibitors, topisomerase inhibitors, and tyrosine kinase inhibitors.

47. The method of claim 43, wherein the one or more chemotherapeutic agents have increased cell permeability when co-administered with vitamin A palmitate compared to administration of the one or more chemotherapeutic agents without vitamin A palmitate.

48. A pharmaceutical composition comprising:
an active pharmaceutical ingredient (API), wherein the API undergoes UGT2B7-mediated glucuronidation; and
a UGT2B7 inhibitor selected from the group consisting of gum rosin and abietic acid.

49. The pharmaceutical composition of claim 48, wherein the API and the UGT2B7 inhibitor are co-formulated as a mixture.

50. The pharmaceutical composition of claim 48, wherein the UGT2B7 inhibitor is not used as a coating.

51. A pharmaceutical composition comprising a chemotherapeutic agent and vitamin A palmitate.

52. The pharmaceutical composition of claim 51, wherein the chemotherapeutic agent is a P-gp substrate.
